(19) United States
(12) Patent Application Publication
(10) Pub. No.: US 2006/0263808 A1
(43) Pub. Date: Nov. 23, 2006

(54) C-met mutations in lung cancer

(75) Inventor: Robert L. Yauch, Redwood City, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 11/388,773

(22) Filed: Mar. 24, 2006

Related U.S. Application Data
(60) Provisional application No. 60/665,317, filed on Mar. 25, 2005.

(51) Int. Cl. C12Q 1/68

(52) U.S. Cl. 435/6

(57) ABSTRACT

The invention provides methods and compositions useful for detecting mutations in c-met in lung cancer cells.

### RELATED APPLICATIONS

This application is a non-provisional application filed under 37 CFR 1.53(b)(1), claiming priority under 35 USC 119(e) to provisional application No. 60/665,317 filed Mar. 25, 2005, the contents of which are incorporated herein by reference.

### TECHNICAL FIELD

The present invention relates generally to the fields of molecular biology and growth factor regulation. More specifically, the invention concerns methods and compositions useful for diagnosing and treating human lung cancer associated with mutated c-met.

### BACKGROUND

HGF is a mesenchyme-derived pleiotrophic factor with mitogenic, motogenic and morphogenic activities on a number of different cell types. HGF effects are mediated through a specific tyrosine kinase, c-met, and aberrant HGF and c-met expression are frequently observed in a variety of tumors. See, e.g., Maulik et al., Cytokine & Growth Factor Reviews (2002), 13:41-59; Danilkovitch-Miagkova & Zbar, J. Clin. Invest. (2002), 109(7):863-867. Regulation of the HGF/c-Met signaling pathway is implicated in tumor progression and metastasis. See, e.g., Trusolino & Comoglio, Nature Rev. (2002), 2:289-300).

HGF binds the extracellular domain of the Met receptor tyrosine kinase (RTK) and regulates diverse biological processes such as cell scattering, proliferation, and survival. HGF-Met signaling is essential for normal embryonic development especially in migration of muscle progenitor cells and development of the liver and nervous system (Bladt et al., Nature (1995), 376, 768-771.; Hamanoue et al., Faseb J (2000), 14, 399-406; Maina et al., Cell (1996), 87, 531-542; Schmidt et al., Nature (1995), 373, 699-702; Uehara et al., Nature (1995), 373, 702-705). Developmental phenotypes of Met and HGF knockout mice are very similar suggesting that HGF is the cognate ligand for the Met receptor (Schmidt et al., 1995, supra; Uehara et al., 1995, supra). HGF-Met also plays a role in liver regeneration, angiogenesis, and wound healing (Bussolino et al., J Cell Biol (1992), 119, 629-641; Matsumoto and Nakamura, Exs (1993), 65, 225-249; Nusrat et al., J Clin Invest (1994) 93; 2056-2065). The precursor Met receptor undergoes proteolytic cleavage into an extracellular α subunit and membrane spanning β subunit linked by disulfide bonds (Tempest et al., Br J Cancer (1988), 58, 3-7). The β subunit contains the cytoplasmic kinase domain and harbors a multi-substrate docking site at the C-terminus where adapter proteins bind and initiate signaling (Bardelli et al., Oncogene (1997), 15, 3103-3111; Nguyen et al., J Biol Chem (1997), 272, 20811-20819; Pelicci et al., Oncogene (1995), 10, 1631-1638; Ponzetto et al., Cell (1994), 77, 261-271; Weidner et al., Nature (1996), 384, 173-176). Upon HGF binding, activation of Met leads to tyrosine phosphorylation and downstream signaling through Gab1 and Grb2/Sos mediated PI3-kinase and Ras/MAPK activation respectively, which drives cell motility and proliferation (Furge et al., Oncogene (2000), 19, 5582-5589; Hartmann et al., J Biol Chem (1994), 269, 21936-21939; Ponzetto et al., J Biol Chem (1996), 271, 14119-14123; Royal and Park, J Biol Chem (1995), 270, 27780-27787).

Met was shown to be transforming in a carcinogen-treated osteosarcoma cell line (Cooper et al., Nature (1984), 311, 29-33; Park et al., Cell (1986), 45, 895-904). Met overexpression or gene-amplification has been observed in a variety of human cancers. For example, Met protein is overexpressed at least 5-fold in colorectal cancers and reported to be gene-amplified in liver metastasis (Di Renzo et al., Clin Cancer Res (1995), 1, 147-154; Liu et al., Oncogene (1992), 7, 181-185). Met protein is also reported to be overexpressed in oral squamous cell carcinoma, hepatocellular carcinoma, renal cell carcinoma, breast carcinoma, and lung carcinoma (Jin et al., Cancer (1997), 79, 749-760; Morello et al., J Cell Physiol (2001), 189, 285-290; Natali et al., Int J Cancer (1996), 69, 212-217; Olivero et al., Br J Cancer (1996), 74, 1862-1868; Suzuki et al., Br J Cancer (1996), 74, 1862-1868). In addition, overexpression of mRNA has been observed in hepatocellular carcinoma, gastric carcinoma, and colorectal carcinoma (Boix et al., Hepatology (1994), 19, 88-91; Kuniyasu et al., Int J Cancer (1993), 55, 72-75; Liu et al., Oncogene (1992), 7, 181-185).

A number of mutations in the kinase domain of Met have been found in renal papillary carcinoma which leads to constitutive receptor activation (Olivero et al., Int J Cancer (1999), 82, 640-643; Schmidt et al., Nat Genet (1997), 16, 68-73; Schmidt et al., Oncogene (1999), 18, 2343-2350). These activating mutations confer constitutive Met tyrosine phosphorylation and result in MAPK activation, focus formation, and tumorigenesis (Jeffers et al., Proc Natl Acad Sci USA (1997), 94, 11445-11450). In addition, these mutations enhance cell motility and invasion (Giordano et al., Faseb J (2000), 14, 399-406; Lorenzato et al., Cancer Res (2002), 62, 7025-7030). HGF-dependent Met activation in transformed cells mediates increased motility, scattering, and migration which eventually leads to invasive tumor growth and metastasis (Jeffers et al., Mol Cell Biol (1996), 16, 1115-1125; Meiners et al., Oncogene (1998), 16, 9-20).

Met has been shown to interact with other proteins that drive receptor activation, transformation, and invasion. In neoplastic cells, Met is reported to interact with α6β4 integrin, a receptor for extracellular matrix (ECM) components such as laminins, to promote HGF-dependent invasive growth (Trusolino et al., Cell (2001), 107, 643-654). In addition, the extracellular domain of Met has been shown to interact with a member of the semaphorin family, plexin B1, and to enhance invasive growth (Giordano et al., Nat Cell Biol (2002), 4, 720-724). Furthermore, CD44v6, which has been implicated in tumorigenesis and metastasis, is also reported to form a complex with Met and HGF and result in Met receptor activation (Orian-Rousseau et al., Genes Dev (2002), 16, 3074-3086).

Met is a member of the subfamily of receptor tyrosine kinases (RTKs) which include Ron and Sea (Maulik et al., Cytokine Growth Factor Rev (2002), 13, 41-59). Prediction of the extracellular domain structure of Met suggests shared homology with the semaphorins and plexins. The N-terminus of Met contains a Sema domain of approximately 500 amino acids that is conserved in all semaphorins and plexins. The semaphorins and plexins belong to a large family of secreted and membrane-bound proteins first described for their role in neural development (Van Vactor and Lorenz, Curr Bio (1999),19, R201-204). However, more recently semaphorin overexpression has been correlated with tumor invasion and metastasis. A cysteine-rich PSI domain (also referred to as a Met Related Sequence domain) found in plexins, semaphorins, and integrins lies adjacent to the Sema domain followed by four IPT repeats that are immunoglobulin-like regions found in plexins and transcription factors. A recent study suggests that the Met Sema domain is sufficient for HGF and heparin binding (Gherardi et al., Proc Natl Acad Sci USA (2003), 100(21):12039-44).

As noted above, the Met receptor tyrosine kinase is activated by its cognate ligand HGF and receptor phosphorylation activates downstream pathways of MAPK, PI-3 kinase and PLC-γ(1, 2). Phosphorylation of Y1234/Y1235 within the kinase domain is critical for Met kinase activation while Y1349 and Y1356 in the multisubstrate docking site are important for binding of src homology-2 (SH2), phosphotyrosine binding (PTB), and Met binding domain (MBD) proteins (3-5), to mediate activation of downstream signaling pathways. An additional juxtamembrane phosphorylation site, Y 1003, has been well characterized for its binding to the tyrosine kinase binding (TKB) domain of the Cb1 E3-ligase (6, 7). Cb1 binding is reported to drive endophilin-mediated receptor endocytosis, ubiquitination, and subsequent receptor degradation (8). This mechanism of receptor downregulation has been described previously in the EGFR family that also harbor a similar Cb1 binding site (9-11).

Dysregulation of Met and HGF have been reported in a variety of tumors. Ligand-driven Met activation has been observed in several cancers. Elevated serum and intra-tumoral HGF is observed in lung, breast cancer, and multiple myeloma (12-15). Overexpression of Met and/or HGF, Met amplification or mutation has been reported in various cancers such as colorectal, lung, gastric, and kidney cancer and is thought to drive ligand-independent receptor activation (2, 16). Additionally, inducible overexpression of Met in a liver mouse model gives rise to hepatocellular carcinoma demonstrating that receptor overexpression drives ligand independent tumorigenesis (17). The most compelling evidence implicating Met in cancer is reported in familial and sporadic renal papillary carcinoma (RPC) patients. Mutations in the kinase domain of Met that lead to constitutive activation of the receptor were identified as germline and somatic mutations in RPC (18). Introduction of these mutations in transgenic mouse models leads to tumorigenesis and metastasis. (19).

Although the role of the Met kinase domain has been investigated in detail, the domains of Met other than the kinase domain is poorly characterized. Indeed, despite being implicated in the etiology of a variety oncological conditions, the HGF/-c-met pathway has been a difficult pathway to target therapeutically. Efforts in this regard have been impeded in large part by the fact that single tumor types are likely to be composed of multiple genetic subtypes, and aberrations of HGF/c-met may constitute only a part of each tumor type. For difficult-to-treat, and genetically and histologically varied cancers such as lung cancers, the problem is particularly acute. Therefore, it is clear that the need for precise methods for identifying cancers that are most likely to respond to inhibition of the HGF/c-met pathway is great. The invention provided herein meets this need and provides other benefits.

All references cited herein, including patent applications and publications, are incorporated by reference in their entirety.

### DISCLOSURE OF THE INVENTION

The present invention is based at least in part on the discovery of multiple mutational events in the receptor of human hepatocyte growth factor (HGF), c-met, that are closely associated with lung tumorigenesis. Although it was previously thought that aberrant c-met activity was associated with various cancers, it was unknown what, if any, specific somatic mutations result in dysregulation of the c-met signaling pathway. In particular, it was not clear what, if any, mutations outside of the kinase domains are associated with the development of human tumors, e.g. lung tumors. It is disclosed herein a variety of mutational events in the extracellular and juxtamembrane domains of c-met that are frequently found in human lung tumors. It is believed that these mutations predispose and/or directly contribute to human lung tumorigenesis. Indeed, as described herein, some of the mutations directly enhance the stability, and consequently amount of c-met protein in human lung tumor cells.

The c-met mutations disclosed herein are useful in a variety of settings, for example in prognostic, predictive, diagnostic, and therapeutic methods and compositions. In one aspect, the invention provides a prognostic method comprising determining whether a lung cancer sample from a subject comprises a mutation in a nucleic acid sequence encoding human c-met, wherein the mutation results in an amino acid change at position N375, I638, V13, V923, I316 and/or E168. In another aspect, the invention provides a prognostic method comprising determining whether a lung cancer sample from a subject comprises a mutation in a nucleic acid sequence encoding human c-met, wherein the sequence is mutated in exon 14 and/or its flanking introns, wherein the mutation affects exon splicing.

In another aspect, the invention provides a method of detecting lung cancer in a sample comprising determining whether the sample comprises a mutation in a nucleic acid sequence encoding human c-met, wherein the mutation results in an amino acid change at position N375, I638, V13, V923, I316 and/or E168. In yet another aspect, the invention provides a method of detecting lung cancer in a sample comprising determining whether the sample comprises a mutation in a nucleic acid sequence encoding human c-met, wherein the mutation is in exon 14 and/or its flanking introns, wherein the mutation affects exon splicing.

In one aspect, the invention provides a method for distinguishing between non-cancerous and cancerous lung tissue, said method comprising determining whether a sample comprising the lung tissue comprises a mutation in a nucleic acid sequence encoding human c-met, wherein the mutation results in an amino acid change at position N375, I638, V13, V923, I316 and/or E168, wherein detection of the mutation in the sample is indicative of presence of cancerous lung tissue. In one aspect, the invention provides a method for distinguishing between non-cancerous and cancerous lung tissue, said method comprising determining whether a sample comprising the lung tissue comprises a mutation in a nucleic acid sequence encoding human c-met, wherein the mutation is in exon 14 and/or its flanking introns, wherein the mutation affects exon splicing, wherein detection of the mutation in the sample is indicative of presence of cancerous lung tissue.

In one aspect, the invention provides a method of identifying a mutation in c-met in lung cancer and/or for detecting a mutated c-met gene in lung cancer, said method comprising contacting a lung cancer sample with an agent capable of detecting a mutation in a nucleic acid sequence encoding human c-met, wherein the mutation results in an amino acid change at position N375, I638, V13, V923, I316 and/or E168. In one aspect, the invention provides a method of identifying a mutation in c-met in lung cancer and/or of detecting a mutated c-met gene in lung cancer, said method comprising contacting a lung cancer sample with an agent capable of detecting a mutation in a nucleic acid sequence encoding human c-met, wherein the mutation is in exon 14 and/or its flanking introns, wherein the mutation affects exon splicing.

In one aspect, the invention provides a method of identifying a lung cancer that is susceptible to treatment with a c-met inhibitor and/or predicting likelihood that a lung cancer will respond to treatment with a c-met inhibitor and/or predicting/identifying which patients diagnosed with lung cancer to treatment with a c-met inhibitor, said method comprising determining whether a lung cancer sample from a subject comprises a mutation in a nucleic acid sequence encoding human c-met, wherein the mutation results in an amino acid change at position N375, I638, V13, V923, I316 and/or E168. In one aspect, the invention provides a method of identifying a lung cancer that is susceptible to treatment with a c-met inhibitor and/or predicting likelihood that a lung cancer will respond to treatment with a c-met inhibitor and/or predicting/identifying which patients diagnosed with lung cancer to treatment with a c-met inhibitor, said method comprising determining whether a lung cancer sample from a subject comprises a mutation in a nucleic acid sequence encoding human c-met, whether the sequence is mutated in exon 14 and/or its flanking introns, wherein the mutation affects exon splicing.

In one aspect, the invention provides a method of determining responsiveness of a lung cancer in a subject to treatment with a c-met inhibitor and/or of monitoring treatment of a subject with a c-met inhibitor, said method comprising determining whether a lung cancer sample from a subject who has been treated with the c-met inhibitor comprises a mutation in a nucleic acid sequence encoding human c-met, wherein the mutation results in an amino acid change at position N375, I638, V13, V923, I316 and/or E168, wherein absence of the mutated nucleic acid sequence is indicative that the lung cancer is responsive to treatment with the c-met inhibitor. In one aspect, the invention provides a method of determining responsiveness of a lung cancer in a subject to treatment with a c-met inhibitor and/or monitoring treatment of a subject with a c-met inhibitor, said method comprising determining whether a lung cancer sample from a subject who has been treated with the c-met inhibitor comprises a mutation in a nucleic acid sequence encoding human c-met, whether the sequence is mutated in exon 14 and/or its flanking introns, wherein the mutation affects exon splicing, wherein absence of the mutated nucleic acid sequence is indicative that the lung cancer is responsive to treatment with the c-met inhibitor.

In one aspect, the invention provides a method for monitoring minimal residual disease in a subject treated for lung cancer with a c-met inhibitor, said method comprising determining whether a sample from a subject who is treated with the c-met inhibitor comprises a mutation in a nucleic acid sequence encoding human c-met, wherein the mutation results in an amino acid change at position N375, I638, V13, V923, I316 and/or E168, wherein detection of said mutation is indicative of presence of minimal residual lung cancer. In one aspect, the invention provides a method for monitoring minimal residual disease in a subject treated for lung cancer with a c-met inhibitor, said method comprising determining whether a lung cancer sample from a subject who has been treated with the c-met inhibitor comprises a mutation in a nucleic acid sequence encoding human c-met, whether the sequence is mutated in exon 14 and/or its flanking introns, wherein the mutation affects exon splicing, wherein detection of said mutation is indicative of presence of minimal residual lung cancer.

In another aspect, the invention provides a method for amplification of a nucleic acid encoding human c-met, wherein the nucleic acid comprises a mutation that results in an amino acid change at position N375, I638, V13, V923, I316 and/or E168 relative to wild type c-met, said method comprising amplifying a sample suspected or known to comprise the nucleic acid with a nucleic acid comprising the sequence of any of the primers/probes listed in Table S4 in FIG. 7. In another aspect, the invention provides a method for amplification of a nucleic acid encoding human c-met, wherein the nucleic acid comprises a mutation in exon 14 and/or its flanking introns, wherein the mutation affects exon splicing, said method comprising amplifying a sample suspected or known to comprise the nucleic acid with a nucleic acid comprising the sequence of any of the primers/probes listed in Table S4 in FIG. 7.

In one aspect, the invention provides a method for identifying a specific mutation in c-met in a sample, wherein the mutation is one that results in an amino acid change at position N375, I638, V13, V923, I316 and/or E168 relative to wild type c-met, said method comprising contacting the sample with a nucleic acid comprising the sequence of any of the primers/probes listed in Table S4 in FIG. 7. In yet another aspect, the invention provides a method for identifying a specific mutation in c-met in a sample, wherein the mutation is in exon 14 and/or its flanking introns, wherein the mutation affects exon splicing, said method comprising contacting the sample with a nucleic acid comprising the sequence of any of the primers/probes listed in Table S4 in FIG. 7.

In one aspect, the invention provides a method of detecting presence of a mutated c-met in lung cancer, the method comprising contacting a sample suspected or known to comprise mutated c-met with a nucleic acid comprising the sequence of any of the primers/probes listed in Table S4 in FIG. 7. In one embodiment, the nucleic acid is hybridized to a nucleic acid probe that is hybridizable to a nucleic acid encoding c-met, and wherein hybridization of the probe is indicative of absence of a mutation in the nucleic acid encoding c-met. In one embodiment, hybridization of the probe is indicative of presence of a mutation in the nucleic acid encoding c-met.

In one aspect, the invention provides a method of detecting presence of a mutated c-met in lung cancer, the method comprising contacting a sample suspected or known to comprise mutated c-met with an antigen binding agent of the invention, wherein binding or lack thereof, of the agent is indicative of presence or absence of a c-met polypeptide comprising a mutation at position N375, I638, V13, V923, I316 and/or E168. In one aspect, the invention provides a method of detecting presence of a mutated c-met in lung cancer, the method comprising contacting a sample suspected or known to comprise mutated c-met with an antigen binding agent of the invention, wherein binding or lack thereof, of the agent is indicative of presence or absence of a c-met polypeptide comprising a deletion of at least a portion of exon 14.

In one aspect, the invention provides a method for detecting a cancerous disease state in a lung tissue, said method comprising determining whether a sample from a subject suspected of having lung cancer comprises a mutation in a nucleic acid sequence encoding human c-met, wherein the mutation results in an amino acid change at position N375, I638, V13, V923, I316 and/or E168, wherein detection of said mutation is indicative of presence of a cancerous disease state in the lung of the subject. In one aspect, the invention provides a method for detecting a cancerous disease state in a lung tissue, said method comprising determining whether a sample from a subject suspected of having lung cancer comprises a mutation in a nucleic acid sequence encoding human c-met, whether the sequence is mutated in exon 14 and/or its flanking introns, wherein the mutation affects exon splicing, wherein detection of said mutation is indicative of presence of minimal residual lung cancer.

The invention also provides a variety of compositions useful for detection and/or diagnosis of lung cancer comprising a c-met mutation as set forth herein. Accordingly, in one aspect, the invention provides a lung cancer biomarker, wherein the biomarker comprises c-met comprising a mutation that results in an amino acid change at position N375, I638, V13, V923, I316 and/or E168. In one aspect, the invention provides a lung cancer biomarker, wherein the biomarker comprises c-met comprising a mutation in exon 14 and/or its flanking introns, wherein the mutation affects exon splicing. Biomarkers of the invention can be in any form that provides information regarding presence or absence of a mutation of the invention. For example, in one embodiment, the biomarker is a nucleic acid molecule. In another embodiment, the biomarker is a polypeptide. In one embodiment, the polypeptide is detectable by an antigen binding agent of the invention that binds to a mutant c-met binding site comprising a mutation site, wherein the mutation site is an amino acid substitution at position N375, I638, V13, V923, I316 and/or E168 or wherein the mutation site is a deleted portion of exon 14. In one embodiment, the deleted portion comprises substantially all of exon 14.

In one aspect, the invention provides a lung cancer imaging agent, wherein the agent specifically binds c-met comprising a mutation, wherein the agent binds a c-met polypeptide comprising a mutation at position N375, I638, V13, V923, I316 and/or E168 of the protein, or wherein the agent binds a c-met encoding nucleic acid comprising a mutation at a nucleic acid position corresponding to a change in amino acid at position N375, I638, V13, V923, I316 and/or E168. In one aspect, the invention provides a lung cancer imaging agent, wherein the agent specifically binds c-met polypeptide comprising a deletion of at least a portion of exon 14, or wherein the agent specifically binds c-met encoding nucleic acid that lacks at least a portion of the sequence that encodes exon 14.

The invention also provides a polynucleotide capable of specifically hybridizing to c-met encoding nucleic acid comprising a mutation at a nucleic acid position corresponding to a change in amino acid at position N375, I638, V13, V923, I316 and/or E168. In another example, the invention provides a polynucleotide capable of specifically hybridizing to c-met encoding nucleic acid that lacks at least a portion of the sequence that encodes exon 14.

In one aspect, the invention provides an antigen binding agent capable of specifically binding to a c-met polypeptide comprising a mutation at position N375, I638, V13, V923, I316 and/or E168. In another aspect, the invention provides an antigen binding agent capable of specifically binding to a c-met polypeptide comprising a deletion of at least a portion of exon 14.

In one aspect, the invention provides an isolated and purified nucleic acid molecule comprising at least a portion of a sequence encoding human c-met, wherein said at least a portion comprises a mutation at a nucleotide position corresponding to a change in c-met amino acid position N375, I638, V13, V923, I316 and/or E168. In another aspect, the invention provides an isolated and purified nucleic acid molecule comprising at least a portion of a genomic sequence encoding human c-met, wherein said at least a portion comprises a mutation in a sequence encoding exon 14 and/or its flanking introns, wherein the mutation affects exon splicing. In one aspect, the invention provides a polypeptide encoded by the nucleic acid molecule of the invention. In one aspect, the invention provides a recombinant vector comprising a nucleic acid molecule of the invention. In one aspect, the invention provides a host cell comprising a recombinant vector of the invention. In one aspect, the invention provides a method of producing a polypeptide of the invention, said method comprising culturing a host cell comprising a recombinant vector of the invention, and isolating the polypeptide expressed from the recombinant vector.

In one aspect, the invention provides an array/gene chip/gene set comprising polynucleotides capable of specifically hybridizing to c-met encoding nucleic acid comprising a mutation at a nucleic acid position corresponding to a change in amino acid at position N375, I638, V13, V923, I316 and/or E168. In another aspect, the invention provides an array/gene chip/gene set comprising polynucleotides capable of specifically hybridizing to c-met encoding nucleic acid that lacks at least a portion of the sequence that encodes exon 14.

In one aspect, the invention provides a computer-readable medium comprising human c-met amino acid polypeptide sequence comprising a mutation at position N375, I638, V13, V923, I316 and/or E168, and/or nucleic acid sequence encoding a human c-met polypeptide comprising a mutation at a nucleic acid position corresponding to a change in amino acid at position N375, I638, V13, V923, I316 and/or E168. In another aspect, the invention provides a computer-readable medium comprising human c-met amino acid polypeptide sequence comprising a deletion of at least a portion of exon 14, and/or human c-met encoding nucleic acid that lacks at least a portion of the sequence that encodes exon 14. In one embodiment, a computer-readable medium of the invention comprises a storage medium for sequence information for one or more subjects. In one embodiment, the information is a personalized genomic profile for a subject known or suspected to have lung cancer, wherein the genomic profile comprises sequence information for c-met comprising a mutation of the invention.

In one aspect, the invention provides a kit and/or article of manufacture comprising a composition of the invention as set forth hereinabove, and instructions for using the composition to detect mutation in human c-met at position N375, I638, V13, V923, I316 and/or E168. In one aspect, the invention provides a kit and/or article of manufacture comprising a composition of the invention, and instructions for using the composition to detect human c-met comprising a deletion of exon 14.

As shown herein, a subset of human lung cancer cells exhibit a deletion of at least a portion of exon 14 of human c-met due to a somatic mutation that results in a hitherto unknown functional human c-met splice variant with oncogenic activity. Accordingly, in one embodiment, a mutation of the invention that affects exon splicing is one that is associated with production of a mutated but functional c-met protein that lacks at least a portion of exon 14. By “functional” is meant that the protein is capable of at least one of the cell signaling activities normally associated with wild-type human c-met protein. In one embodiment, the portion of exon 14 that is deleted results in removal of the Y1003 phosphorylation site necessary for Cb1 binding and down regulation of the activated c-met receptor. In one embodiment, a mutant c-met comprising deletion of at least a portion of exon 14 comprises substantially intact exon 13 and exon 15. In one embodiment, a mutant c-met comprising deletion of at least a portion of exon 14 comprises transmembrane domain and/or extracellular domain of wild-type c-met. In one embodiment, a mutant c-met comprising deletion of at least a portion of exon 14 comprises an extracellular ligand binding domain. Somatic mutations capable of affecting exon splicing in the manner hereindescribed can be determined by one skilled in the art based on the examples set forth herein. Examples of such mutations include any mutation(s) that is associated with a change in the splicing machinery normally associated with human c-met RNA splicing. For example, such mutations include one or more sequence alterations in the 5′ or 3′ splice sites, the branch point, polypyrimidine tract, etc., such as those set forth in FIG. 1A, FIG. 2, and Table S3 in FIG. 6. Further confirmation of presence or production of a functional c-met splice variant can be determined using techniques known in the art, some of which are described in the Examples below.

In one embodiment, a mutation at position N375, I638, V13, V923, I316 and/or E168 results in these substitutions, respectively: N375S, I638L, V13L, V923L, I316M, and E168D. Specific substitutions are also indicated in Table S3 of FIG. 6.

A mutation of the invention can be detected by any suitable method known in the art, including but not limited to a) restriction-fragment-length-polymorphism detection based on allele-specific restriction-endonuclease cleavage, (b) hybridization with allele-specific oligonucleotide probes including immobilized oligonucleotides or oligonucleotide arrays, (c) allele-specific PCR, mismatch-repair detection (MRD), (d) binding of MutS protein, (e) denaturing-gradient gel electrophoresis (DGGE), (f) single-strand-conformation-polymorphism detection, (g) RNAase cleavage at mismatched base-pairs, (h) chemical or enzymatic cleavage of heteroduplex DNA, (i) methods based on allele specific primer extension, (j) genetic bit analysis (GBA), (k) oligonucleotide-ligation assay (OLA), (l) allele-specific ligation chain reaction (LCR), (m) gap-LCR, and (n) radioactive, colorimetric and/or fluorescent DNA sequencing.

### BRIEF DESCRIPTION OF THE DRAWINGS

FIG. 1. Identification of tumor-specific, intronic mutations in Met leading to exon 14 splicing. (A) A schematic representation of Met exon 14 showing the position of 3 identified nucleic acid deletions &/or point mutations (solid lines &/or arrowhead) with respect to the splice site junctions (based on RefSeq, NM_{—}000245). H596, cell line; pat. 14./pat. 16, patient tumor specimens. (B) RT-PCR amplification of the RNA transcript encompassing exon 14 from specimens harboring either intronic mutations or wild-type Met. WT, wild-type; U, unspliced; S, spliced. (C) Met protein expression in lysates from patient-matched, normal lung tissue and primary tumor tissue from specimens expressing wild-type or mutant Met transcripts. Actin immunoblots serve as a protein loading control. Total Met transcript levels were assessed by quantitative PCR and relative expression valures are indicated (2^{-ΔCt}). Abbreviations: N, normal lung tissue; T, primary lung tumor. (D) Schematic representation of the Met protein showing the distribution of identified amino acid alterations from either primary lung tumor specimens (upper) or lung cell lines and xenograft models (lower). Amino acid deletions are shown as bars and substitutions as arrowheads. Genetic alterations were confirmed as somatic mutations (black bars/arrowhead), polymorphisms (white arrowheads), or not determined (grey bars/arrowheads), based on genomic DNA sequencing of patient-matched, non-neoplastic, lung tissue.

FIG. 2 depicts illustrative intronic mutations flanking exon 14 of Met. A schematic representation of Met exon 14 showing the corresponding nucleic acid (NM_{—}000245) deletions and/or point mutations (light grey text) with respect to the intron/exon structure. (A) H596, lung cancer cell line. (B) pat. 14, patient 14 lung tumor specimen. (C) pat. 16, patient 16 lung tumor specimen. For reference, for tumor H596, there is a point mutation from G to T at position marked +1 in (A). For tumor Pat 14, there is a deletion of the sequence from position marked −27 to −6 in (B). For tumor Pat 16, there is a deletion of the sequence from position marked 3195 to +7 in (C). Representative sequencing chromatograms in both the sense and antisense directions are also shown.

FIG. 3. Intronic mutations are absent in non-neoplastic lung tissue from patient 14 and 16. Sequencing chromatograms in both the sense (F) and antisense (R) directions highlighting the position of the corresponding deletions (black brackets) from patients 14 (A) and 16 (B). Black arrows represent the positions of either the 5′ or 3′ splice junctions flanking exon 14.

FIG. 4. Table S1 showing a summary of lung and colon cancer specimens sequenced.

FIG. 5. Table S2 showing a summary of Met and K-ras genetic alterations in lung and colon cancer specimens.

FIG. 6. Table S3 showing a detailed synopsis of specimens with Met genetic alterations.

FIG. 7. Table S4 depicting PCR primers used for sequencing.

FIG. 8 depicts illustrative cis-acting splicing elements expected to regulate splicing of human c-met exon 14. It is expected that a mutation at one or more positions within these elements would have a negative impact on wild type splicing of exon 14.

FIG. 9 depicts wild-type human c-met protein sequence based on RefSeq. NM_{—}000245.

### MODES FOR CARRYING OUT THE INVENTION

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Primers, oligonucleotides and polynucleotides employed in the present invention can be generated using standard techniques known in the art.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

Definitions

The term “array” or “microarray”, as used herein refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane. The nucleotide sequences can be DNA, RNA, or any permutations thereof.

A “target sequence”, “target nucleic acid” or “target protein”, as used herein, is a polynucleotide sequence of interest, in which a mutation of the invention is suspected or known to reside, the detection of which is desired. Generally, a “template,” as used herein, is a polynucleotide that contains the target nucleotide sequence. In some instances, the terms “target sequence,” “template DNA,” “template polynucleotide,” “target nucleic acid,” “target polynucleotide,” and variations thereof, are used interchangeably.

“Amplification,” as used herein, generally refers to the process of producing multiple copies of a desired sequence. “Multiple copies” mean at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc. ), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

A “primer” is generally a short single stranded polynucleotide, generally with a free 3′-OH group, that binds to a target potentially present in a sample of interest by hybridizing with a target sequence, and thereafter promotes polymerization of a polynucleotide complementary to the target.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

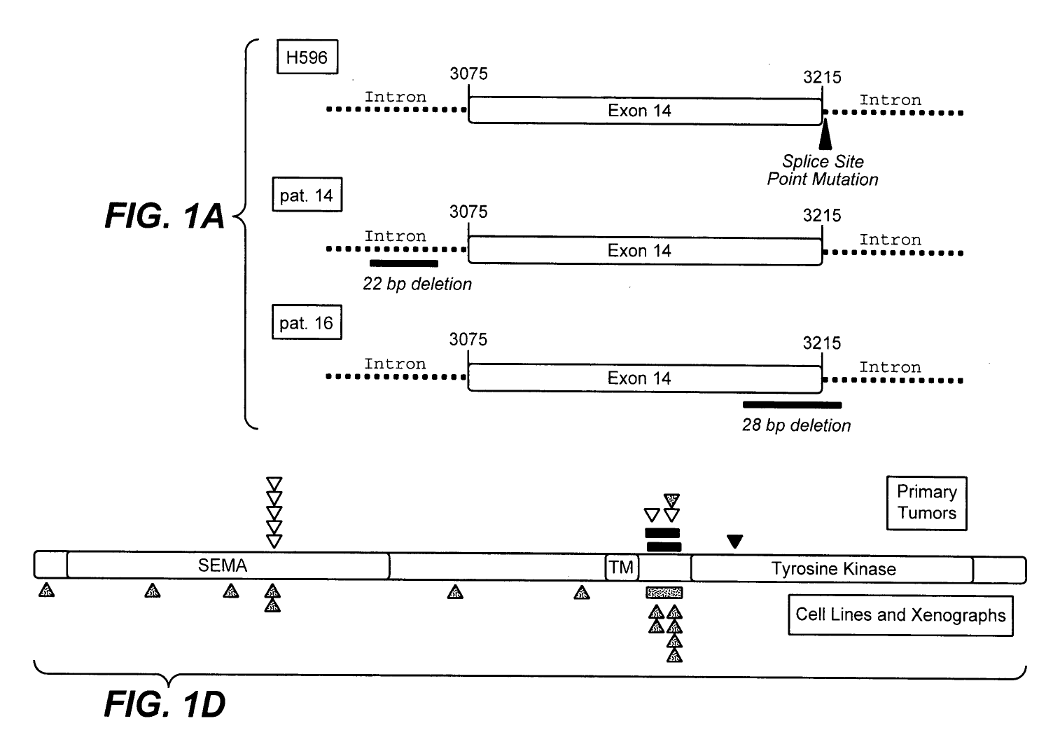

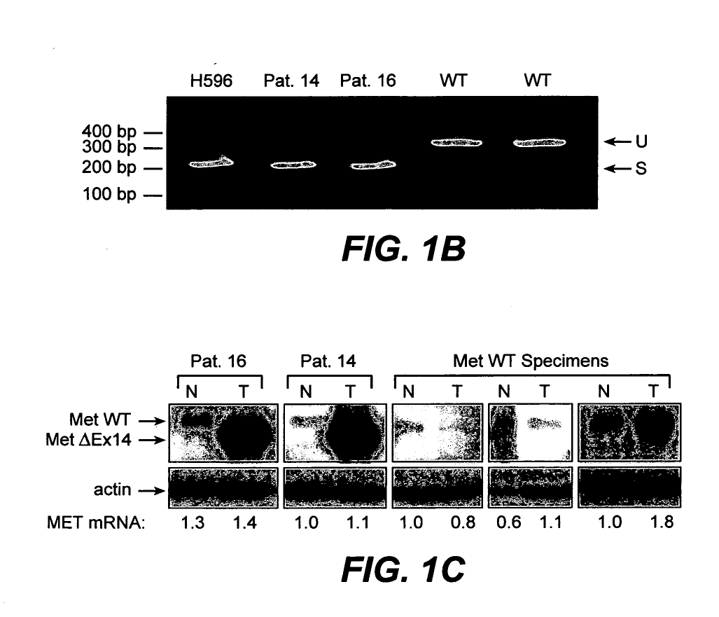

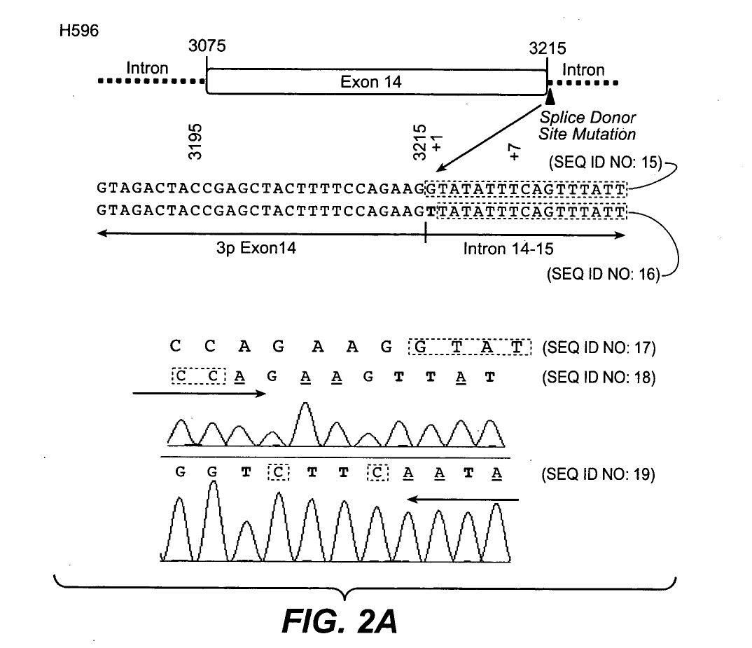

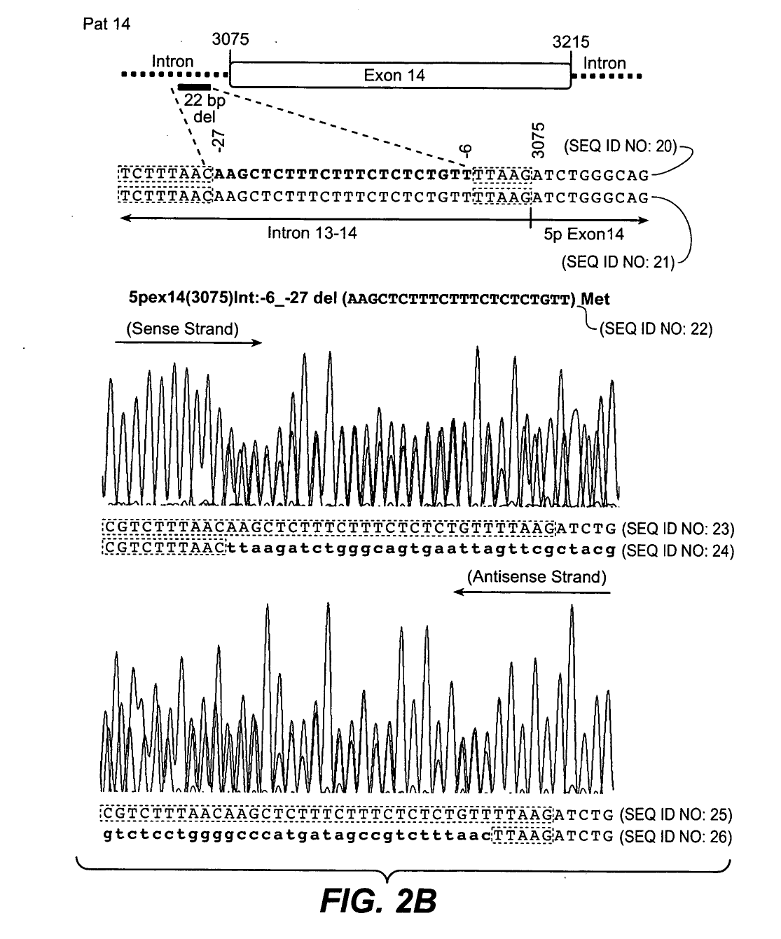

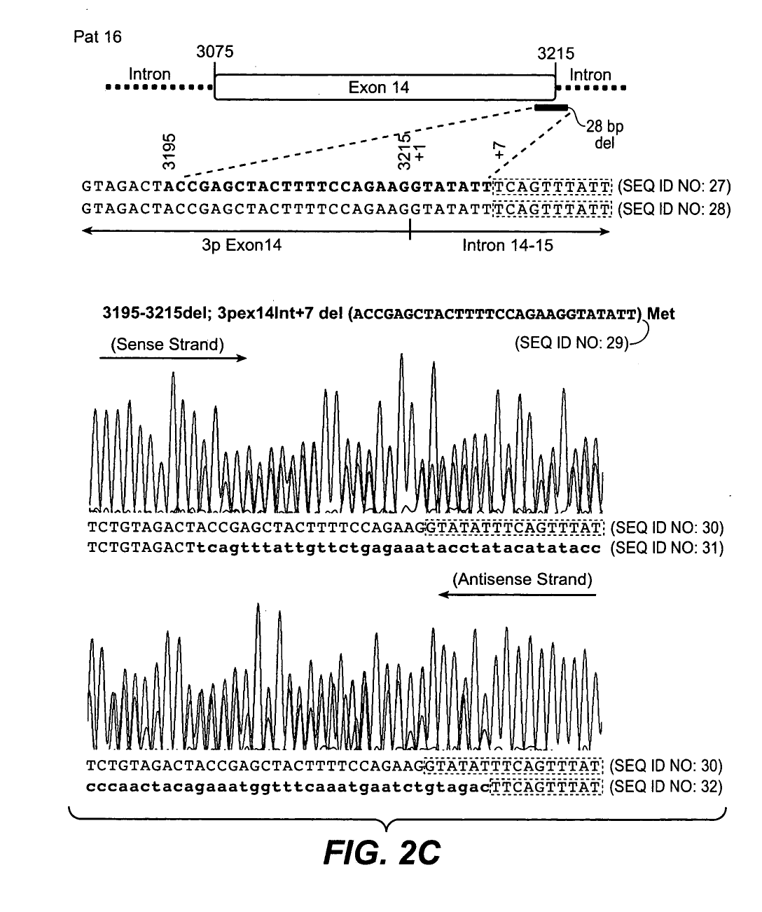

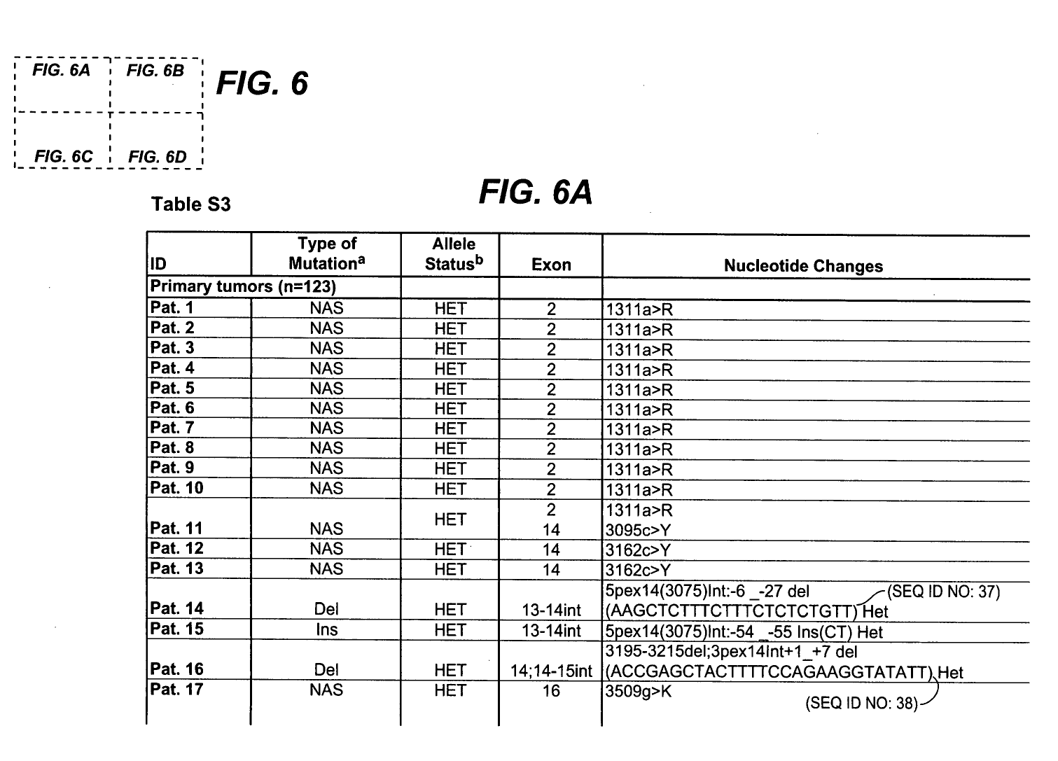

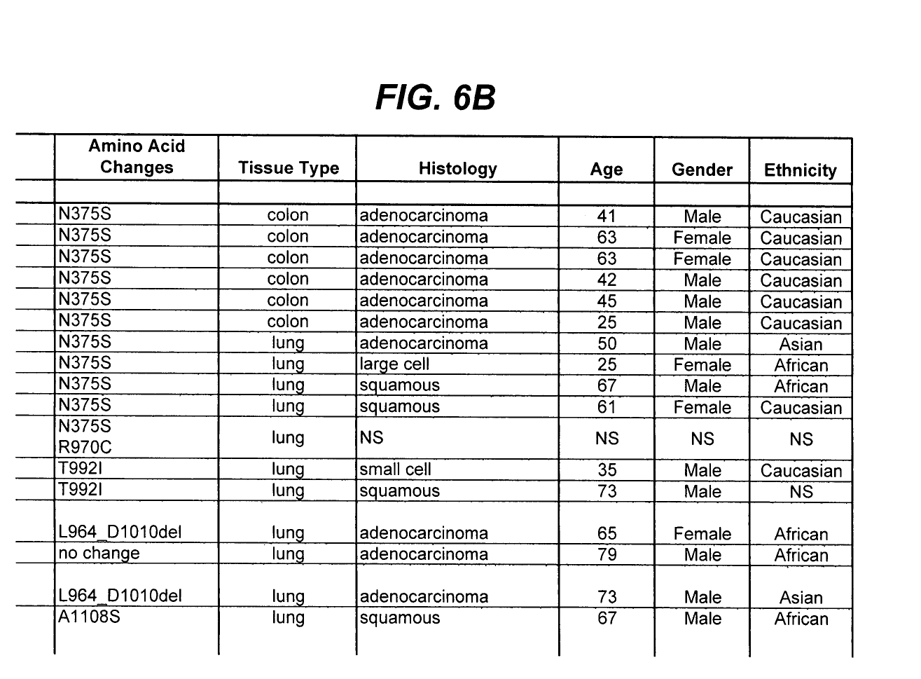

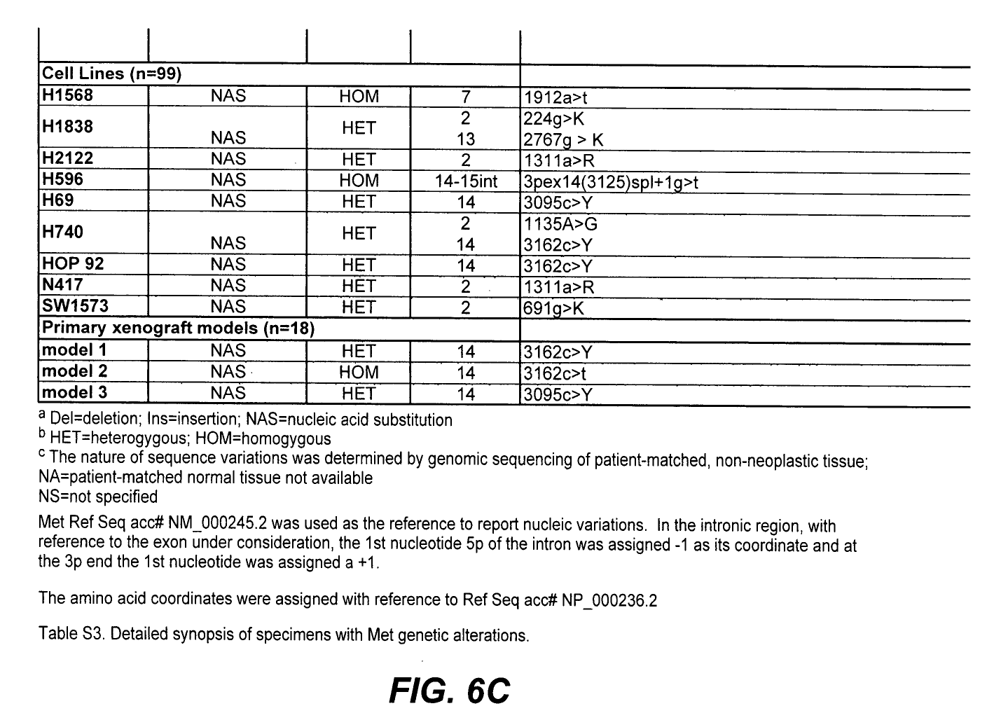

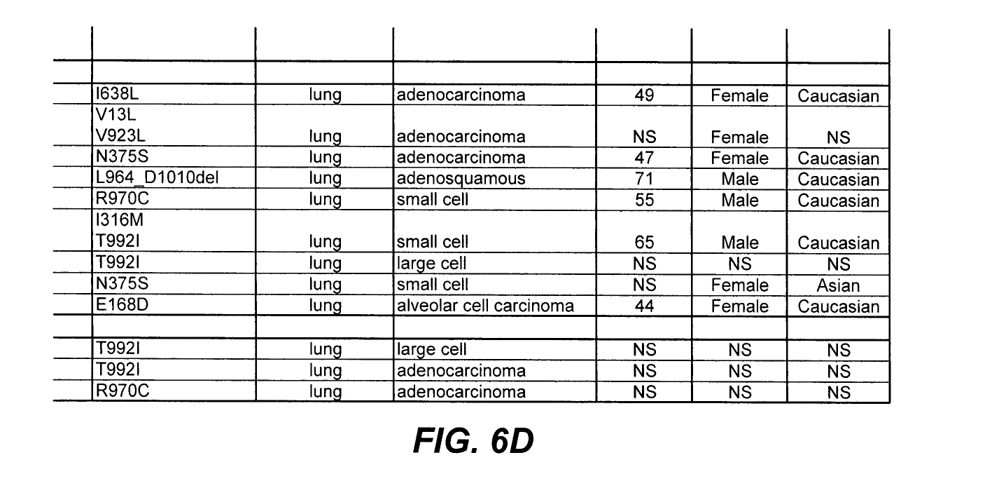

The term “mutation”, as used herein, means a difference in the amino acid or nucleic acid sequence of a particular protein or nucleic acid (gene, RNA) relative to the wild-type protein or nucleic acid, respectively. A mutated protein or nucleic acid can be expressed from or found on one allele (heterozygous) or both alleles (homozygous) of a gene, and may be somatic or germ line. In the instant invention, mutations are generally somatic. In a particular embodiment, said mutation is found outside of the kinase domain region (KDR) of c-met, for example in the extracellular domain or juxtamembrane domain. In another embodiment the mutation is an amino acid substitution, deletion or insertion as shown in Table S3 in FIG. 6, FIG. 1A, FIG. 2. Mutations include sequence rearrangements such as insertions, deletions, and point mutations (including single nucleotide/amino acid polymorphisms).

To “inhibit” is to decrease or reduce an activity, function, and/or amount as compared to a reference.

The term “3”′ generally refers to a region or position in a polynucleotide or oligonucleotide 3′ (downstream) from another region or position in the same polynucleotide or oligonucleotide. Thus, for example, a 3′ splice site in reference to an exon is located downstream from the 5′ end of that exon. Similarly, a 3′ splice site in reference to an intron is located downstream from the 5′ end of that intron.

The term “5”′ generally refers to a region or position in a polynucleotide or oligonucleotide 5′ (upstream) from another region or position in the same polynucleotide or oligonucleotide. Thus, for example, a 5′ splice site in reference to an exon is located upstream from the 3′ end of that exon. Similarly, a 5′ splice site in reference to an intron is located upstream from the 3′ end of that intron.

“Detection” includes any means of detecting, including direct and indirect detection.

The term “diagnosis” is used herein to refer to the identification of a molecular or pathological state, disease or condition, such as the identification of a lung cancer. The term “prognosis” is used herein to refer to the prediction of the likelihood of lung cancer-attributable death or progression, including, for example, recurrence, metastatic spread, and drug resistance, of a neoplastic disease, such as lung cancer. The term “prediction” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The term “long-term” survival is used herein to refer to survival for at least 1 year, 5 years, 8 years, or 10 years following therapeutic treatment.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase-in the length of survival following treatment; and/or (9) decreased mortality at a given point of time following treatment.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The terms “c-met inhibitor” and “c-met antagonist”, as used herein, refer to a molecule having the ability to inhibit a biological function of wild type or mutated c-met. Accordingly, the term “inhibitor” is defined in the context of the biological role of c-met. In one embodiment, a c-met inhibitor referred to herein specifically inhibits cell signaling via the HGF/c-met pathway. For example, a c-met inhibitor may interact with (e.g. bind to) c-met, or with a molecule that normally binds to c-met. In one embodiment, a c-met inhibitor binds to the extracellular domain of c-met. In one embodiment, a c-met inhibitor binds to the intracellular domain of c-met. In one embodiment, c-met biological activity inhibited by a c-met inhibitor is associated with the development, growth, or spread of a tumor. A c-met inhibitor can be in any form, so long as it is capable of inhibiting HGF/c-met activity; inhibitors include antibodies (e.g., monoclonal antibodies as defined hereinbelow), small organic/inorganic molecules, antisense oligonucleotides, aptamers, inhibitory peptides/polypeptides, inhibitory RNAs (e.g., small interfering RNAs), combinations thereof, etc.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “hypervariable region”, “HVR”, or “HV”, when used herein refers to the regions of an antibody variable domain which are hypervariable in sequence and/or form structurally defined loops. The letters “HC” and “LC” preceding the term “HVR” or “HV” refers, respectively, to HVR or HV of a heavy chain and light chain. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

“Framework” or “FR” residues are those variable domain residues other than the hypervariable region residues as herein defined.

The “variable region” or “variable domain” of an antibody refers to the amino-terminal domains of heavy or light chain of the antibody. These domains are generally the most variable parts of an antibody and contain the antigen-binding sites.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs/HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR/HVR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The Fc region of an immunoglobulin generally comprises two constant domains, a CH2 domain and a CH3 domain, and optionally comprises a CH4 domain. By “Fc region chain” herein is meant one of the two polypeptide chains of an Fc region.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; amninopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

A “blocking” antibody or an “antagonist” antibody is one which inhibits or reduces biological activity of the antigen it binds. Such blocking can occur by any means, e.g. by interfering with protein-protein interaction such as ligand binding to a receptor. In on embodiment, blocking antibodies or antagonist antibodies substantially or completely inhibit the biological activity of the antigen.

The terms “cancer” and “cancerous” refer to or describe the physiological condition in mammals that is typically characterized by unregulated cell growth/proliferation. Examples of cancer include but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer. Methods and compositions of the invention are particularly useful for, and are generally directed to human lung cancer, including for example non-small cell lung cancer and small cell lung cancer, which can be histologically characterized as an adenocarcinoma, large cell, squamous, small cell, an alveolar cell carcinoma, adenosquamous, etc.

The term “tumor,” as used herein, refers to all neoplastic cell growth and proliferation, whether malignant or benign, and all pre-cancerous and cancerous cells and tissues.

The term “sample”, as used herein, refers to a composition that is obtained or derived from a subject of interest that contains a cellular and/or other molecular entity that is to be characterized and/or identified based on, for example, physical, biochemical, chemical and/or physiological characteristics. For example, the phrase “lung cancer sample” or “lung tumor sample” refers to any sample obtained from a subject of interest that would be expected or is known to contain the cellular and/or molecular entity that is to be characterized.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods and compositions of the invention are useful in attempts to delay development of a disease or disorder.

An “effective amount” refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

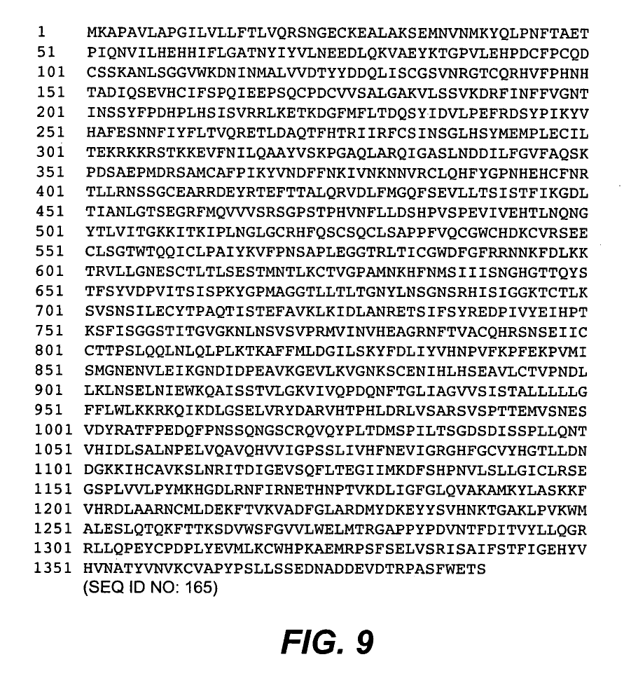

The term “hepatocyte growth factor” or “HGF”, as used herein, refers, unless indicated otherwise, to any native or variant (whether native or synthetic) HGF polypeptide that is capable of activating the HGF/c-met signaling pathway under conditions that permit such process to occur. The term “wild type HGF” generally refers to a polypeptide comprising the amino acid sequence of a naturally occurring HGF protein. Thet term “wild type HGF sequence” generally refers to an amino acid sequence found in a naturally occurring HGF. C-met is a known receptor for HGF through which HGF intracellular signaling is biologically effectuated. A wild type human c-met protein sequence based on RefSeq NM_{—}000245 is depicted in FIG. 9.

The term “housekeeping gene” refers to a group of genes that codes for proteins whose activities are essential for the maintenance of cell function. These genes are typically similarly expressed in all cell types. Housekeeping genes include, without limitation, glyceraldehyde-3-phosphate dehydrogenase (GAPDH), Cyp1, albumin, actins, e.g. β-actin, tubulins, cyclophilin, hypoxantine phsophoribosyltransferase (HRPT), L32. 28S, and 18S.

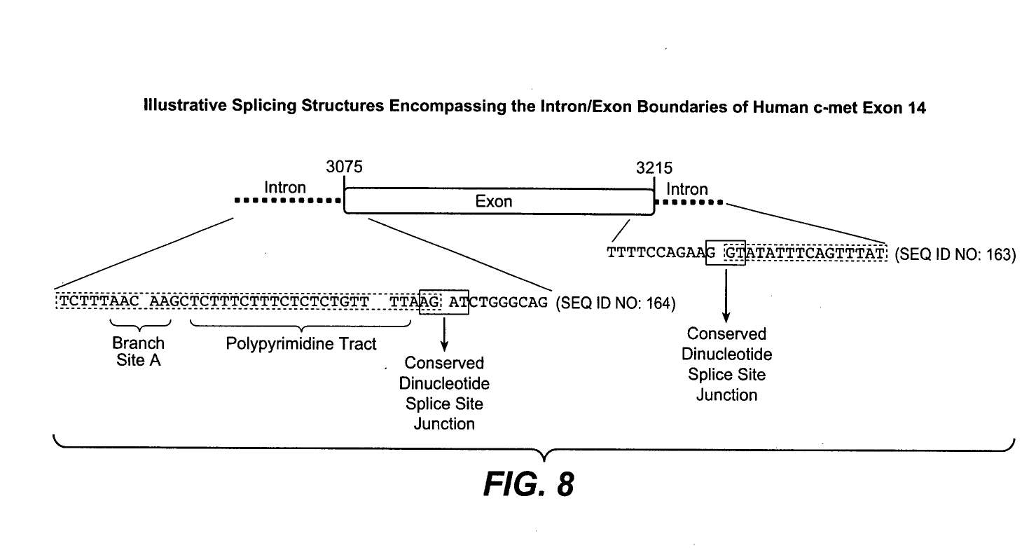

The terms “splice site”, “splice junction”, “branch point”, “polypyrimidine tract”, as used herein, refer to the meaning known in the art in the context of mammalian, in particular human, RNA splicing. See, e.g., Pagani & Baralle, Nature Reviews: Genetics (2004), 5:389-396, and references cited therein. For convenient reference, one embodiment of sequences for c-met RNA splicing elements is illustratively set forth in FIG. 8.

General Illustrative Techniques

Methods for detection of nucleic acid mutations are well known in the art. Often, though not necessarily, a target nucleic acid in a sample is amplified to provide the desired amount of material for determination of whether a mutation is present. Amplification techniques are well known in the art. For example, the amplified product may or may not encompass all of the nucleic acid sequence encoding the protein of interest, so long as the amplified product comprises the particular amino acid/nucleic acid sequence position where the mutation is suspected to be.

In one example, presence of a mutation can be determined by contacting nucleic acid from a sample with a nucleic acid probe that is capable of specifically hybridizing to nucleic acid encoding a mutated nucleic acid, and detecting said hybridization. In one embodiment, the probe is detectably labeled, for example with a radioisotope (³H, ³²P, ³³P etc), a fluorescent agent (rhodamine, fluorescene etc.) or a chromogenic agent. In some embodiments, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, nucleic acid probes of the invention are provided in a kit for identifying c-met mutations in a sample, said kit comprising an oligonucleotide that specifically hybridizes to or adjacent to a site of mutation in the nucleic acid encoding c-met. The kit may further comprise instructions for treating patients having tumors that contain c-met mutations with a c-met inhibitor based on the result of a hybridization test using the kit.

Mutations can also be detected by comparing the electrophoretic mobility of an amplified nucleic acid to the electrophoretic mobility of corresponding nucleic acid encoding wild-type c-met. A difference in the mobility indicates the presence of a mutation in the amplified nucleic acid sequence. Electrophoretic mobility may be determined by any appropriate molecular separation technique, for example on a polyacrylamide gel.

Nucleic acids may also be analyzed for detection of mutations using Enzymatic Mutation Detection (EMD) (Del Tito et al, Clinical Chemistry 44:731-739, 1998). EMD uses the bacteriophage resolvase T₄ endonuclease VII, which scans along double-stranded DNA until it detects and cleaves structural distortions caused by base pair mismatches resulting from nucleic acid alterations such as point mutations, insertions and deletions. Detection of two short fragments formed by resolvase cleavage, for example by gel eletrophoresis, indicates the presence of a mutation. Benefits of the EMD method are a single protocol to identify point mutations, deletions, and insertions assayed directly from amplification reactions, eliminating the need for sample purification, shortening the hybridization time, and increasing the signal-to-noise ratio. Mixed samples containing up to a 20-fold excess of normal nucleic acids and fragments up to 4 kb in size can been assayed. However, EMD scanning does not identify particular base changes that occur in mutation positive samples, therefore often requiring additional sequencing procedures to identiity the specific mutation if necessary. CEL I enzyme can be used similarly to resolvase T₄ endonuclease VII, as demonstrated in U.S. Pat. No. 5,869,245.

Another simple kit for detecting the mutations of the invention is a reverse hybridization test strip similar to Haemochromatosis StripAssay™ (Viennalabs http://www.bamburghmarrsh.com/pdf/4220.pdf) for detection of multiple mutations in HFE, TFR2 and FPN1 genes causing Haemochromatosis. Such an assay is based on sequence specific hybridization following amplification by PCR. For single mutation assays, a microplate-based detection system may be applied, whereas for multi-mutation assays, test strips may be used as “macro-arrays” . Kits may include ready-to use reagents for sample prep, amplification and mutation detection. Multiplex amplification protocols provide convenience and allow testing of samples with very limited volumes. Using the straightforward StripAssay format, testing for twenty and more mutations may be completed in less than five hours without costly equipment. DNA is isolated from a sample and the target nucleic acid is amplified in vitro (e.g., by PCR) and biotin-labelled, generally in a single (“multiplex”) amplification reaction. The amplification products are then selectively hybridized to oligonucleotide probes (wild-type and mutant specific) immobilized on a solid support such as a test strip in which the probes are immobilized as parallel lines or bands. Bound biotinylated amplicons are detected using streptavidin-alkaline phosphatase and color substrates. Such an assay can detect all or any subset of the mutations of the invention. With respect to a particular mutant probe band, one of three signalling patterns are possible: (i) a band only for wild-type probe which indicates normal nucleic acid sequence, (ii) bands for both wild-type and a mutant probe which indicates heterozygous genotype, and (iii) band only for the mutant probe which indicates homozygous mutant genotype. Accordingly, in one aspect, the invention provides a method of detecting mutations of the invention comprising isolating and/or amplifying a target c-met nucleic acid sequence from a sample, such that the amplification product comprises a ligand, contacting the amplification product with a probe which comprises a detectable binding partner to the ligand and the probe is capable of specifically hydribizing to a mutation of the invention, and then detecting the hybridization of said probe to said amplification product. In one embodiment, the ligand is biotin and the binding partner comprises avidin or streptavidin. In one embodiment, the binding partner comprises steptavidin-alkaline which is detectable with color substrates. In one embodiment, the probes are immobilized for example on a test strip wherein probes complementary to different mutations are separated from one another. Alternatively, the amplified nucleic acid is labelled with a radioisotope in which case the probe need not comprise a detectable label.

According to the methods of the present invention, alteration of the wild-type c-met gene is detected. Alterations of a wild-type gene according to the present invention encompasses all forms of mutations such as insertions, inversions, deletions, and/or point mutations. In one embodiment, the mutations are somatic. Somatic mutations are those which occur only in certain tissues, e.g., in the tumor tissue, and are not inherited in the germ line. Germ line mutations can be found in any of a body's tissues. If only a single allele is somatically mutated, an early neoplastic state is indicated. However, if both alleles are mutated, then a late neoplastic state is indicated. The finding of c-met mutations is therefore a diagnostic and prognostic indicator as described herein.

The c-met mutations found in tumor tissues may result in predisposing cells comprising the mutation, or other cells with which the mutated cells interact, to tumorigenesis. In some instances, it is expected that mutations of the invention are associated with increased signaling activity relative to wild-type c-met, thereby leading to a cancerous state. Indeed, mutations of the invention that lead to deletion of exon 14 result in stabilization of c-met protein, thereby increasing signaling of the c-met pathway and enhancing tumorigenic capabilities of the lung cells comprising the mutations.

A sample comprising a target nucleic acid can be obtained by methods well known in the art, and that are appropriate for the particular type and location of the tumor. Tissue biopsy is often used to obtain a representative piece of tumor tissue. Alternatively, tumor cells can be obtained indirectly in the form of tissues/fluids that are known or thought to contain the tumor cells of interest. For instance, samples of lung cancer lesions may be obtained by resection, bronchoscopy, fine needle aspiration, bronchial brushings, or from sputum, pleural fluid or blood. Mutant genes or gene products can be detected from tumor or from other body samples such as urine, sputum or serum. The same techniques discussed above for detection of mutant target genes or gene products in tumor samples can be applied to other body samples. Cancer cells are sloughed off from tumors and appear in such body samples. By screening such body samples, a simple early diagnosis can be achieved for diseases such as cancer. In addition, the progress of therapy can be monitored more easily by testing such body samples for mutant target genes or gene products.

The methods of the invention are applicable to any tumor in which c-met has a role in tumorigenesis. The diagnostic methods of the present invention are useful for clinicians so that they can decide upon an appropriate course of treatment. For example, a tumor displaying alteration of both target gene alleles might suggest a more aggressive therapeutic regimen than a tumor displaying alteration of only one of the alleles. Methods of the invention can be utilized in a variety of settings, including for example in aiding in patient selection during the course of drug development, prediction of likelihood of success when treating an individual patient with a particular treatment regimen, in assessing disease progression, in monitoring treatment efficacy, in determining prognosis for individual patients, in assessing predisposition of an individual to develop a particular cancer (e.g., lung cancer), in differentiating tumor type and/or tumor staging, etc.

Means for enriching a tissue preparation for tumor cells are known in the art. For example, the tissue may be isolated from paraffin or cryostat sections. Cancer cells may also be separated from normal cells by flow cytometry or laser capture microdissection. These, as well as other techniques for separating tumor from normal cells, are well known in the art. If the tumor tissue is highly contaminated with normal cells, detection of mutations may be more difficult, although techniques for minimizing contamination and/or false positive/negative results are known, some of which are described hereinbelow. For example, a sample may also be assessed for the presence of a biomarker (including a mutation) known to be associated with a tumor cell of interest but not a corresponding normal cell, or vice versa.

Detection of point mutations in target nucleic acids may be accomplished by molecular cloning of the target nucleic acids and sequencing the nucleic acids using techniques well known in the art. Alternatively, amplification techniques such as the polymerase chain reaction (PCR) can be used to amplify target nucleic acid sequences directly from a genomic DNA preparation from the tumor tissue. The nucleic acid sequence of the amplified sequences can then be determined and mutations identified therefrom. Amplification techniques are well known in the art, e.g., polymerase chain reaction as described in Saiki et al., Science 239:487, 1988; U.S. Pat. Nos. 4,683,203 and 4,683,195.

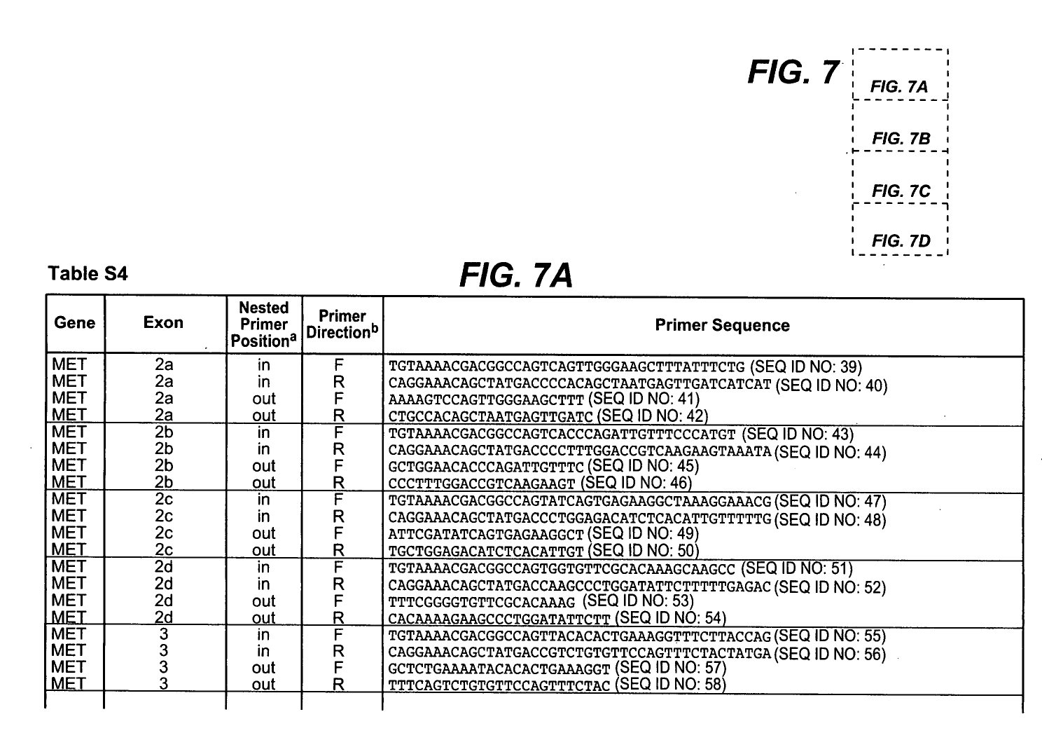

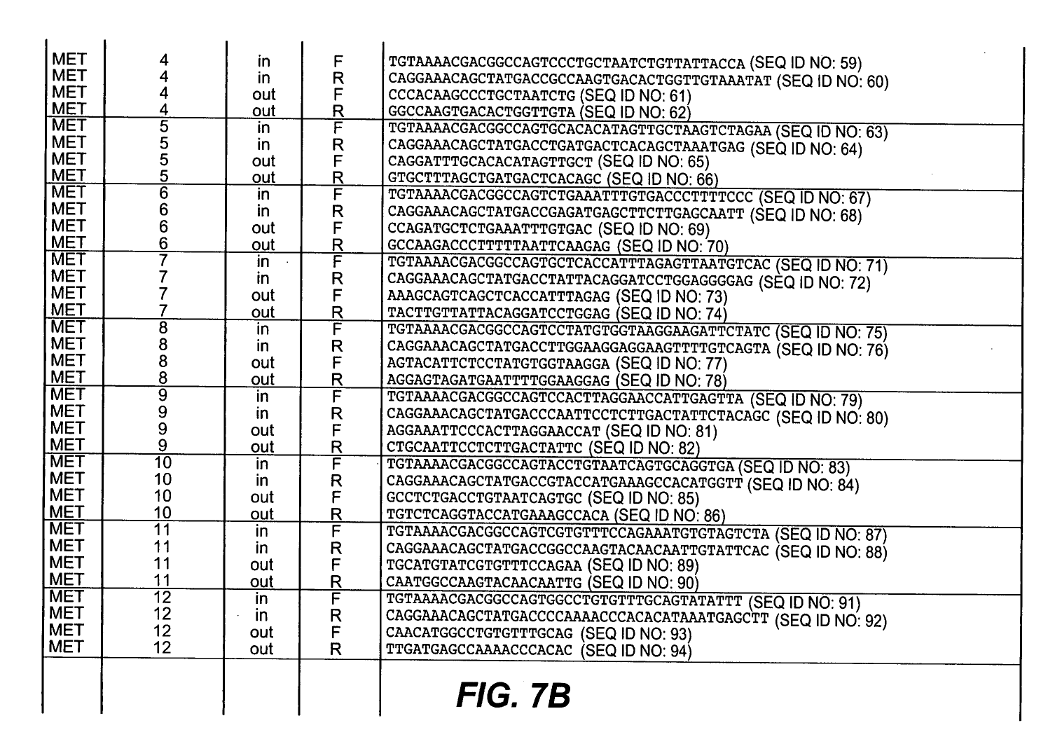

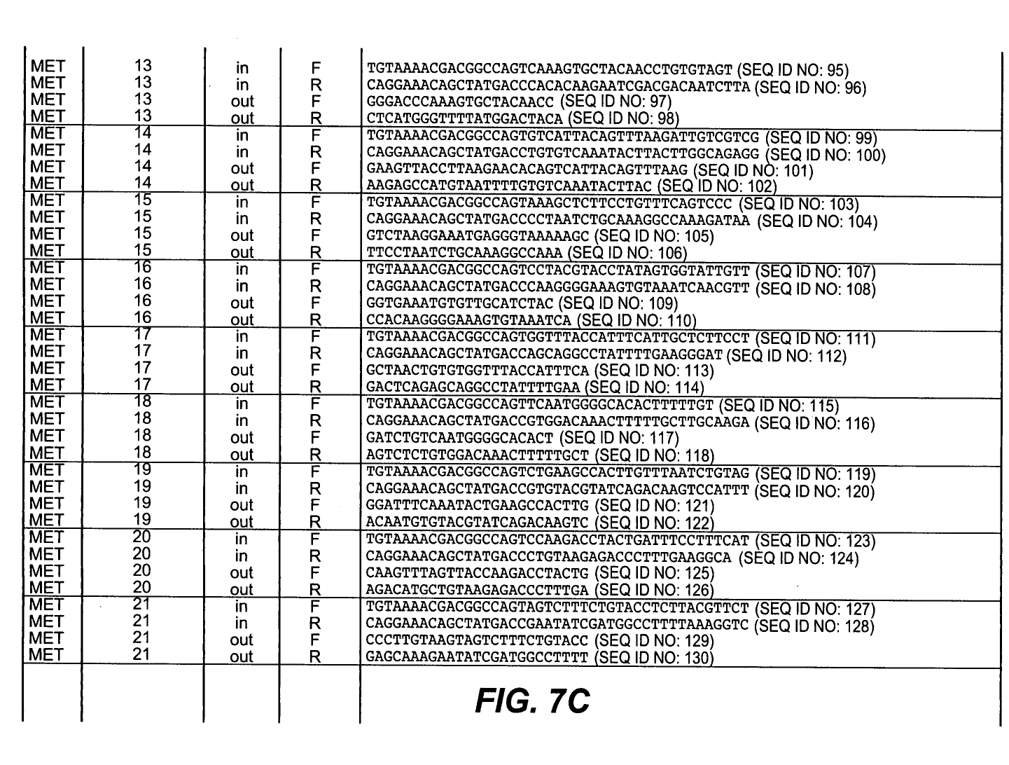

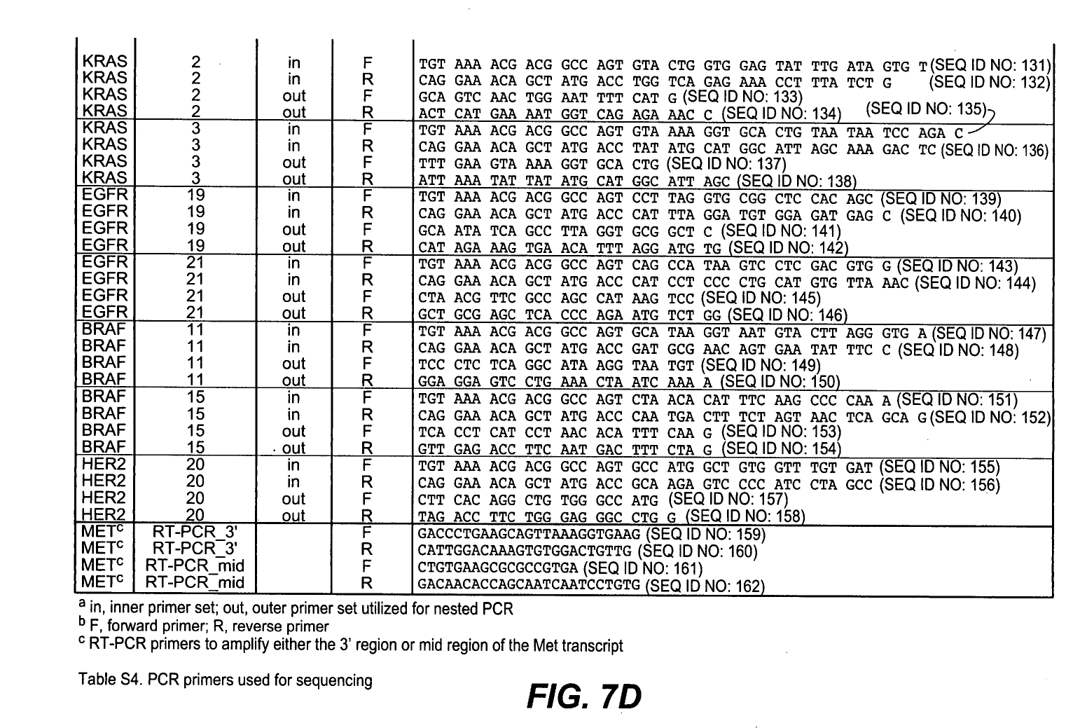

Specific primer pairs which can be used for amplification of target nucleic acids of the invention include those listed in Table S4 in FIG. 7. However, it should be noted that design and selection of appropriate primers are well established techniques in the art, and therefore methods and compositions of the invention comprise the use of any nucleic acid probes/primers designed based on the primers in Table S4 in FIG. 7 and/or the target nucleic acid sequence.

The ligase chain reaction, which is known in the art, can also be used to amplify target nucleic acid sequences. See, e.g., Wu et al., Genouics, Vol. 4, pp. 560-569 (1989). In addition, a technique known as allele specific PCR can also be used. See, e.g., Ruano and Kidd, Nucleic Acids Research, Vol. 17, p. 8392, 1989. According to this technique, primers are used which hybridize at their 3′ ends to a particular target nucleic acid mutation. If the particular mutation is not present, an amplification product is not observed. Amplification Refractory Mutation System (ARMS) can also be used, as disclosed in European Patent Application Publication No. 0332435, and in Newton et al., Nucleic Acids Research, Vol. 17, p. 7, 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Single stranded conformation polymorphism (SSCP) analysis can also be used to detect base change variants of an allele. See, e.g. Orita et al., Proc. Natl. Acad. Sci. USA Vol. 86, pp. 2766-2770, 1989, and Genomics, Vol. 5, pp. 874-879, 1989. Other techniques for detecting insertions and deletions as known in the art can also be used.

Alteration of wild-type genes can also be detected on the basis of the alteration of a wild-type expression product of the gene. Such expression products include both mRNA as well as the protein product. Point mutations may be detected by amplifying and sequencing the mRNA or via molecular cloning of cDNA made from the mRNA. The sequence of the cloned cDNA can be determined using DNA sequencing techniques which are well known in the art. The cDNA can also be sequenced via the polymerase chain reaction (PCR).

Mismatches, according to the present invention, are hybridized nucleic acid duplexes which are not 100% complementary. The lack of total complementarity may be due to deletions, insertions, inversions, substitutions or frameshift mutations. Mismatch detection can be used to detect point mutations in a target nucleic acid. While these techniques can be less sensitive than sequencing, they are simpler to perform on a large number of tissue samples. An example of a mismatch cleavage technique is the RNase protection method, which is described in detail in Winter et al., Proc. Natl. Acad. Sci. USA, Vol. 82, p. 7575, 1985, and Meyers et al., Science, Vol. 230, p. 1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid mRNA or gene, but can a portion of the target nucleic acid, provided it encompasses the position suspected of being mutated. If the riboprobe comprises only a segment of the target nucleic acid mRNA or gene, it may be desirable to use a number of these probes to screen the whole target nucleic acid sequence for mismatches if desired.

In a similar manner, DNA probes can be used to detect mismatches, for example through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the target nucleic acid mRNA or DNA which might contain a mutation can be amplified before hybridization. Changes in target nucleic acid DNA can also be detected using Southern hybridization, especially if the changes are gross rearrangements, such as deletions and insertions.

Target nucleic acid DNA sequences which have been amplified may also be screened using allele-specific probes. These probes are nucleic acid oligomers, each of which contains a region of the target nucleic acid gene harboring a known mutation. For example, one oligomer may be about 30 nucleotides in length, corresponding to a portion of the target gene sequence. By use of a battery of such allele-specific probes, target nucleic acid amplification products can be screened to identify the presence of a previously identified mutation in the target gene. Hybridization of allele-specific probes with amplified target nucleic acid sequences can be performed, for example, on a nylon filter. Hybridization to a particular probe under stringent hybridization conditions indicates the presence of the same mutation in the tumor tissue as in the allele-specific probe.

Alteration of wild-type target genes can also be detected by screening for alteration of the corresponding wild-type protein. For example, monoclonal antibodies immunoreactive with a target gene product can be used to screen a tissue, for example an antibody that is known to bind to a particular mutated position of the gene product (protein). For example, an antibody that is used may be one that binds to a deleted exon (e.g., exon 14) or that binds to a conformational epitope comprising a deleted portion of the target protein. Lack of cognate antigen would indicate a mutation. Antibodies specific for products of mutant alleles could also be used to detect mutant gene product. Antibodies may be identified from phage display libraries. Such immunological assays can be done in any convenient format known in the art. These include Western blots, immunohistochemical assays and ELISA assays. Any means for detecting an altered protein can be used to detect alteration of wild-type target genes.

The primer pairs of the present invention are useful for determination of the nucleotide sequence of a target nucleic acid using nucleic acid amplification techniques such as the polymerase chain reaction. The pairs of single stranded DNA primers can be annealed to sequences within or surrounding the target nucleic acid sequence in order to prime amplification of the target sequence. Allele-specific primers can also be used. Such primers anneal only to particular mutant target sequence, and thus will only amplify a product in the presence of the mutant target sequence as a template. In order to facilitate subsequent cloning of amplified sequences, primers may have restriction enzyme site sequences appended to their ends. Such enzymes and sites are well known in the art. The primers themselves can be synthesized using techniques which are well known in the art. Generally, the primers can be made using oligonucleotide synthesizing machines which are commercially available. Design of particular primers is well within the skill of the art.

The nucleic acid probes provided by the invention are useful for a number of purposes. They can be used in Southern hybridization to genomic DNA and in the RNase protection method for detecting point mutations already discussed above. The probes can be used to detect target nucleic acid amplification products. They may also be used to detect mismatches with the wild type gene or mRNA using other techniques. Mismatches can be detected using either enzymes (e.g., S1 nuclease), chemicals (e.g., hydroxylamine or osmium tetroxide and piperidine), or changes in electrophoretic mobility of mismatched hybrids as compared to totally matched hybrids. These techniques are known in the art. See Novack et al., Proc. Natl. Acad. Sci. USA, Vol. 83, p. 586, 1986. Generally, the probes are complementary to sequences outside of the kinase domain. An entire battery of nucleic acid probes may be used to compose a kit for detecting mutations in target nucleic acids. The kit allows for hybridization to a large region of a target sequence of interest. The probes may overlap with each other or be contiguous.

If a riboprobe is used to detect mismatches with mRNA, it is generally complementary to the mRNA of the target gene. The riboprobe thus is an antisense probe in that it does not code for the corresponding gene product because it is complementary to the sense strand. The riboprobe generally will be labeled with a radioactive, colorimetric, or fluorometric material, which can be accomplished by any means known in the art. If the riboprobe is used to detect mismatches with DNA it can be of either polarity, sense or anti-sense. Similarly, DNA probes also may be used to detect mismatches.

The invention also provides a variety of compositions suitable for use in performing methods of the invention. For example, the invention provides arrays that can be used in such methods. In one embodiment, an array of the invention comprises individual or collections of nucleic acid molecules useful for detecting mutations of the invention. For instance, an array of the invention may comprises a series of discretely placed individual nucleic acid oligonucleotides or sets of nucleic acid oligonucleotide combinations that are hybridizable to a sample comprising target nucleic acids, whereby such hybridization is indicative of presence or absence of a mutation of the invention.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into nucleic acid molecules that are synthesized. The synthesized product is then contacted with a solid substrate, such as a glass slide, which is coated with an aldehyde or another reactive group which will form a covalent link with the reactive group that is on the amplified product and become covalently attached to the glass slide. Other methods, such as those using amino propryl silican surface chemistry are also known in the art, as disclosed at http://www.cmt.corning.com and http://cmgm.standord.ecu/pbrown1.

Attachment of groups to oligonucleotides which could be later converted to reactive groups is also possible using methods known in the art. Any attachment to nucleotides of oligonucleotides will become part of oligonucleotide, which could then be attached to the solid surface of the microarray.

Amplified nucleic acids can be further modified, such as through cleavage into fragments or by attachment of detectable labels, prior to or following attachment to the solid substrate, as required and/or permitted by the techniques used.

In some methods of the invention, an antigen binding agent that binds specifically to c-met comprising a mutation of the invention but not wild type c-met is used. Such agent be any suitable binding agent, such as antibodies, binder polypeptides and aptamers. Generation of such binding agents are known in the art, and described in, e.g., U.S. Pat. Appl. Pub. No. 2005/0042216.

Examples of c-met Inhibitor Antibodies

Examples of c-met inhibitor antibodies include c-met inhibitors that interfere with binding of a ligand such as HGF to c-met. For example, a c-met inhibitor may bind to c-met such that binding of HGF to c-met is inhibited. In one embodiment, an antagonist antibody is a chimeric antibody, for example, an antibody comprising antigen binding sequences from a non-human donor grafted to a heterologous non-human, human or humanized sequence (e.g., framework and/or constant domain sequences). In one embodiment, the non-human donor is a mouse. In one embodiment, an antigen binding sequence is synthetic, e.g. obtained by mutagenesis (e.g., phage display screening, etc.). In one embodiment, a chimeric antibody of the invention has murine V regions and human C region. In one embodiment, the murine light chain V region is fused to a human kappa light chain. In one embodiment, the murine heavy chain V region is fused to a human IgG1 C region. In one embodiment, the antigen binding sequences comprise at least one, at least two or all three CDRs of a light and/or heavy chain. In one embodiment, the antigen binding sequences comprise a heavy chain CDR3. In one embodiment, the antigen binding sequences comprise part or all of the CDR and/or variable domain sequences of the monoclonal antibody produced by the hybridoma cell line deposited under American Type Culture Collection Accession Number ATCC HB-11894 (hybridoma 1A3.3.13) or HB-11895 (hybridoma 5D5.11.6). In one embodiment, the antigen binding sequences comprise at least CDR3 of the heavy chain of the monoclonal antibody produced by the hybridoma cell line 1A3.3.13 or 5D5.11.6. Humanized antibodies of the invention include those that have amino acid substitutions in the FR and affinity maturation variants with changes in the grafted CDRs. The substituted amino acids in the CDR or FR are not limited to those present in the donor or recipient antibody. In other embodiments, the antibodies of the invention further comprise changes in amino acid residues in the Fc region that lead to improved effector function including enhanced CDC and/or ADCC function and B-cell killing. Other antibodies of the invention include those having specific changes that improve stability. Antibodies of the invention also include fucose deficient variants having improved ADCC function in vivo.

In one embodiment, an antibody fragment of the invention comprises an antigen binding arm comprising a heavy chain comprising at least one, at least two or all three of CDR sequences selected from the group consisting of SYWLH (SEQ ID NO:1), MIDPSNSDTRFNPNFKD (SEQ ID NO:2) and YGSYVSPLDY (SEQ ID NO:3). In one embodiment, the antigen binding arm comprises heavy chain CDR-H1 having amino acid sequence SYWLH. In one embodiment, the antigen binding arm comprises heavy chain CDR-H2 having amino acid sequence MIDPSNSDTRFNPNFKD. In one embodiment, the antigen binding arm comprises heavy chain CDR-H3 having amino acid sequence YGSYVSPLDY. In one embodiment, an antibody fragment of the invention comprises an antigen binding arm comprising a light chain comprising at least one, at least two or all three of CDR sequences selected from the group consisting of KSSQSLLYTSSQKNYLA (SEQ ID NO:4), WASTRES (SEQ ID NO:5) and QQYYAYPWT (SEQ ID NO:6). In one embodiment, the antigen binding arm comprises heavy chain CDR-L1 having amino acid sequence KSSQSLLYTSSQKNYLA. In one embodiment, the antigen binding arm comprises heavy chain CDR-L2 having amino acid sequence WASTRES. In one embodiment, the antigen binding arm comprises heavy chain CDR-L3 having amino acid sequence QQYYAYPWT. In one embodiment, an antibody fragment of the invention comprises an antigen binding arm comprising a heavy chain comprising at least one, at least two or all three of CDR sequences selected from the group consisting of SYWLH (SEQ ID NO:1), MIDPSNSDTRFNPNFKD (SEQ ID NO:2) and YGSYVSPLDY (SEQ ID NO:3) and a light chain comprising at least one, at least two or all three of CDR sequences selected from the group consisting of KSSQSLLYTSSQKNYLA (SEQ ID NO:4), WASTRES (SEQ ID NO:5) and QQYYAYPWT (SEQ ID NO:6).

The invention provides a humanized antagonist antibody that binds human c-met, or an antigen-binding fragment thereof, wherein the antibody is effective to inhibit human HGF/c-met activity in vivo, the antibody comprising in the H chain Variable region (V_{H}) at least a CDR3 sequence of the monoclonal antibody produced by the hybridoma cell line deposited under American Type Culture Collection Accession Number ATCC HB-11894 (hybridoma 1A3.3.13) or HB-11895 (hybridoma 5D5.11.6) and substantially a human consensus sequence (e.g., substantially the human consensus framework (FR) residues of human heavy chain subgroup III (V_{H}III)). In one embodiment, the antibody further comprises the H chain CDR1 sequence and/or CDR2 sequence of the monoclonal antibody produced by the hybridoma cell line deposited under American Type Culture Collection Accession Number ATCC HB-11894 (hybridoma 1A3.3.13) or HB-11895 (hybridoma 5D5.11.6). In another embodiment, the preceding antibody comprises the L chain CDR1 sequence, CDR2 sequence and/or CDR3 sequence of the monoclonal antibody produced by the hybridoma cell line deposited under American Type Culture Collection Accession Number ATCC HB-11894 (hybridoma 1A3.3.13) or HB-11895 (hybridoma 5D5.11.6) with substantially the human consensus framework (FR) residues of human light chain κ subgroup I (VκI).

In one embodiment, an antibody fragment of the invention comprises an antigen binding arm comprising a heavy chain variable domain having the sequence:

(SEQ ID NO:7)

QVQLQQSGPELVRPGASVKMSCRASGYTFTSYWLHWVKQRPGQGLEWIGM
IDPSNSDTRFNPNFKDKATLNVDRSSNTAYMLLSSLTSADSAVYYCATYG
SYVSPLDYWGQGTSVTVSS

In one embodiment, an antibody fragment of the invention comprises an antigen binding arm comprising a light chain variable domain having the sequence:

(SEQ ID NO:8)

DIMMSQSPSSLTVSVGEKVTVSCKSSQSLLYTSSQKNYLAWYQQKPGQSP
KLLIYWASTRESGVPDRFTGSGSGTDFTLTITSVKADDLAVYYCQQYYAY
PWTFGGGTKLEIK

Yet in other instances, it may be advantageous to have a c-met antagonist that does not interfere with binding of a ligand (such as HGF) to c-met. Accordingly, in some embodiments, an antagonist of the invention does not bind a ligand (such as HGF) binding site on c-met. In another embodiment, an antagonist of the invention does not substantially inhibit ligand (e.g., HGF) binding to c-met. In one embodiment, an antagonist of the invention does not substantially compete with a ligand (e.g., HGF) for binding to c-met. In one example, an antagonist of the invention can be used in conjunction with one or more other antagonists, wherein the antagonists are targeted at different processes and/or functions within the HGF/c-met axis. Thus, in one embodiment, a c-met antagonist of the invention binds to an epitope on c-met distinct from an epitope to which another c-met antagonist, such as the Fab fragment of the monoclonal antibody produced by the hybridoma cell line deposited under American Type Culture Collection Accession Number ATCC HB-11894 (hybridoma 1A3.3.13) or HB-11895 (hybridoma 5D5.11.6), binds. In another embodiment, a c-met antagonist of the invention is distinct from (i.e., it is not) a Fab fragment of the monoclonal antibody produced by the hybridoma cell line deposited under American Type Culture Collection Accession Number ATCC HB-11894 (hybridoma 1A3.3.13) or HB-11895 (hybridoma 5D5.11.6). In one embodiment, a c-met antagonist of the invention does not comprise a c-met binding sequence of an antibody produced by the hybridoma cell line deposited under American Type Culture Collection Accession Number ATCC HB-11894 (hybridoma 1A3.3.13) or HB-11895 (hybridoma 5D5.11.6). In one embodiment, an antagonist of the invention inhibits c-met activity but does not bind to a wild-type juxtamembrane domain of c-met.

C-met antagonist antibodies of the invention can be any antibody that is capable of interfering with c-met activity. Some specific examples include an anti-c-met antibody comprising:

(a) at least one, two, three, four or five hypervariable region (HVR) sequences selected from the group consisting of:

(i) HVR-L1 comprising sequence A1-A17, wherein A1-A17 is KSSQSLLYTSSQKNYLA (SEQ ID NO:1)

(ii) HVR-L2 comprising sequence B1-B7, wherein B1-B7 is WASTRES (SEQ ID NO:2)

(iii) HVR-L3 comprising sequence C1-C9, wherein C1-C9 is QQYYAYPWT (SEQ ID NO:3)

(iv) HVR-H1 comprising sequence D1-D10, wherein D1-D10 is GYTFTSYWLH (SEQ ID NO:4)

(v) HVR-H2 comprising sequence E1-E18, wherein E1-E18 is GMIDPSNSDTRFNPNFKD (SEQ ID NO:5) and

(vi) HVR-H3 comprising sequence F1-F11, wherein F1-F11 is XYGSYVSPLDY (SEQ ID NO:6) and X is not R;

and (b) at least one variant HVR, wherein the variant HVR sequence comprises modification of at least one residue of the sequence depicted in SEQ ID NOs:1, 2, 3, 4, 5 or 6. In one embodiment, HVR-L1 of an antibody of the invention comprises the sequence of SEQ ID NO:1. In one embodiment, HVR-L2 of an antibody of the invention comprises the sequence of SEQ ID NO:2. In one embodiment, HVR-L3 of an antibody of the invention comprises the sequence of SEQ ID NO:3. In one embodiment, HVR-H1 of an antibody of the invention comprises the sequence of SEQ ID NO:4. In one embodiment, HVR-H2 of an antibody of the invention comprises the sequence of SEQ ID NO:5. In one embodiment, HVR-H3 of an antibody of the invention comprises the sequence of SEQ ID NO:6. In one embodiment, HVR-H3 comprises TYGSYVSPLDY (SEQ ID NO:7). In one embodiment, HVR-H3 comprises SYGSYVSPLDY (SEQ ID NO:8). In one embodiment, an antibody of the invention comprising these sequences (in combination as described herein) is humanized or human.

In one aspect, the invention provides an antibody comprising one, two, three, four, five or six HVRs, wherein each HVR comprises, consists or consists essentially of a sequence selected from the group consisting of SEQ ID NOs: 1, 2, 3, 4, 5, 6, 7, and 8, and wherein SEQ ID NO:1 corresponds to an HVR-L1, SEQ ID NO:2 corresponds to an HVR-L2, SEQ ID NO:3 corresponds to an HVR-L3, SEQ ID NO:4 corresponds to an HVR-H1, SEQ ID NO:5 corresponds to an HVR-H2, and SEQ ID NOs:6, 7 or 8 corresponds to an HVR-H3. In one embodiment, an antibody of the invention comprises HVR-L1, HVR-L2, HVR-L3, HVR-H1, HVR-H2, and HVR-H3, wherein each, in order, comprises SEQ ID NO:1, 2, 3, 4, 5 and 7. In one embodiment, an antibody of the invention comprises HVR-L1, HVR-L2, HVR-L3, HVR-H1, HVR-H2, and HVR-H3, wherein each, in order, comprises SEQ ID NO:1, 2, 3, 4, 5 and 8.

Variant HVRs in an antibody of the invention can have modifications of one or more residues within the HVR. In one embodiment, a HVR-L2 variant comprises 1-5 (1, 2, 3, 4 or 5) substitutions in any combination of the following positions: B1 (M or L), B2 (P, T, G or S), B3 (N, G, R or T), B4 (I, N or F), B5 (P, I, L or G), B6 (A, D, T or V) and B7 (R, I, M or G). In one embodiment, a HVR-H1 variant comprises 1-5 (1, 2, 3, 4 or 5) substitutions in any combination of the following positions: D3 (N, P, L, S, A, I), D5 (I, S or Y), D6 (G, D, T, K, R), D7 (F, H, R, S, T or V) and D9 (M or V). In one embodiment, a HVR-H2 variant comprises 1-4 (1, 2, 3 or 4) substitutions in any combination of the following positions: E7 (Y), E9 (I), E10 (I), E14 (T or Q), E15 (D, K, S, T or V), E16 (L), E17 (E, H, N or D) and E18 (Y, E or H). In one embodiment, a HVR-H3 variant comprises 1-5 (1, 2, 3, 4 or 5) substitutions in any combination of the following positions: F1 (T, S), F3 (R, S, H, T, A, K), F4 (G), F6 (R, F, M, T, E, K, A, L, W), F7 (L, I, T, R, K, V), F8 (S, A), F10 (Y, N) and F11 (Q, S, H, F). Letter(s) in parenthesis following each position indicates an illustrative substitution (i.e., replacement) amino acid; as would be evident to one skilled in the art, suitability of other amino acids as substitution amino acids in the context described herein can be routinely assessed using techniques known in the art and/or described herein. In one embodiment, a HVR-L1 comprises the sequence of SEQ ID NO:1. In one embodiment, F1 in a variant HVR-H3 is T. In one embodiment, F1 in a variant HVR-H3 is S. In one embodiment, F3 in a variant HVR-H3 is R. In one embodiment, F3 in a variant HVR-H3 is S. In one embodiment, F7 in a variant HVR-H3 is T. In one embodiment, an antibody of the invention comprises a variant HVR-H3 wherein F1 is T or S, F3 is R or S, and F7 is T.

In one embodiment, an antibody of the invention comprises a variant HVR-H3 wherein F1 is T, F3 is R and F7 is T. In one embodiment, an antibody of the invention comprises a variant HVR-H3 wherein F1 is S. In one embodiment, an antibody of the invention comprises a variant HVR-H3 wherein F1 is T, and F3 is R. In one embodiment, an antibody of the invention comprises a variant HVR-H3 wherein F1 is S, F3 is R and F7 is T. In one embodiment, an antibody of the invention comprises a variant HVR-H3 wherein F1 is T, F3 is S, F7 is T, and F8 is S. In one embodiment, an antibody of the invention comprises a variant HVR-H3 wherein F1 is T, F3 is S, F7 is T, and F8 is A. In some embodiments, said variant HVR-H3 antibody further comprises HVR-L1, HVR-L2, HVR-L3, HVR-H1 and HVR-H2 wherein each comprises, in order, the sequence depicted in SEQ ID NOs:1, 2, 3, 4 and 5. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In one embodiment, an antibody of the invention comprises a variant HVR-L2 wherein B6 is V. In some embodiments, said variant HVR-L2 antibody further comprises HVR-L1, HVR-L3, HVR-H1, HVR-H2 and HVR-H3, wherein each comprises, in order, the sequence depicted in SEQ ID NOs:1, 3, 4, 5 and 6. In some embodiments, said variant HVR-L2 antibody further comprises HVR-L1, HVR-L3, HVR-H1, HVR-H2 and HVR-H3, wherein each comprises, in order, the sequence depicted in SEQ ID NOs:1, 3, 4, 5 and 7. In some embodiments, said variant HVR-L2 antibody further comprises HVR-L1, HVR-L3, HVR-H1, HVR-H2 and HVR-H3, wherein each comprises, in order, the sequence depicted in SEQ ID NOs:1, 3, 4, 5 and 8. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In one embodiment, an antibody of the invention comprises a variant HVR-H2 wherein E14 is T, E15 is K and E17 is E. In one embodiment, an antibody of the invention comprises a variant HVR-H2 wherein E17 is E. In some embodiments, said variant HVR-H3 antibody further comprises HVR-L1, HVR-L2, HVR-L3, HVR-H1, and HVR-H3 wherein each comprises, in order, the sequence depicted in SEQ ID NOs:1, 2, 3, 4 and 6. In some embodiments, said variant HVR-H2 antibody further comprises HVR-L1, HVR-L2, HVR-L3, HVR-H1, and HVR-H3, wherein each comprises, in order, the sequence depicted in SEQ ID NOs:1, 2, 3, 4, and 7. In some embodiments; said variant HVR-H2 antibody further comprises HVR-L1, HVR-L2, HVR-L3, HVR-H1, and HVR-H3, wherein each comprises, in order, the sequence depicted in SEQ ID NOs:1, 2, 3, 4, and 8. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

The following are examples of the methods and compositions of the invention. It is understood that various other embodiments may be practiced, given the general description provided above.

### EXAMPLES

### Materials and Methods

Sequence Analysis

Frozen primary tumor tissue specimens were stained with hematoxylin and eosin (H&E) to confirm diagnosis and evaluate tumor content. Specimens exhibiting >50% tumor content were selected for DNA extraction. PCR amplifications of genomic DNA were carried out using nested primers (Table S4) and products were purified using ExoSAP-IT kit (USB). PCR products were subsequently sequenced in both sense and antisense directions. For confirmation of nucleotide deletions, PCR products were TOPO cloned and 3-5 individual clones were sequenced. For sequencing of cDNA products, RNA was amplified using Qiagen's One-Step RT-PCR kit.

Quantitative PCR

Total Met transcript expression levels were assessed by quantitative RT-PCR using standard Taqman techniques. Met transcript levels were normalized to the housekeeping gene, β-glucuronidase (GUS) and results are expressed as normalized expression values (=2^{ΔCt}). The primer/probe sets for GUS was forward, 5′-TGGTTGGAGAGCTCATITGGA-3′; reverse, 5′-GCACTCTCGTCGGTGACTGTT-3′; and probe, 5′(VIC)-TMTGCCGATTTCATGACT-(MGBNFQ)-3′. The primer/probe set for Met was forward, 5′-CATTAAAGGAGACCTCACCATAGCTAAT-3′; reverse, 5′-CCTGATCGAGAAACCACAACCT-3′; and probe, 5-(FAM)-CATGAAGCGACCCTCTGATGTCCCA-(BHQ-1)-3′. The Met amplicon represents a conserved region between wildtype and alternatively spliced Met transcripts.

Cell Culture

Cell lines were obtained from American Type Culture Collection (ATCC), NCI Division of Cancer Treatment and Diagnosis tumor repository, or Japanese Health Sciences Foundation. All cell lines were maintained in RPMI 1640 supplemented with 10% FBS (Sigma), penicillin/streptomycin (GIBCO), and 2 mM L-glutamine.

Western Blot Analysis

For protein expression analyses in frozen tissue specimens, tissue (˜100 mg) was homogenized in 200 μl of cell lysis buffer (Cell Signaling), containing protease inhibitor cocktail (Sigma), phosphatase inhibitor cocktails I and II (Sigma), 50 mM sodium fluoride, and 2 mM sodium orthovanadate using a Polytron® homogenizer (Kinematica). Samples were further lysed by gentle rocking for 1 hour at 4° C., prior to preclearance with a mixture of Protein A Sepharose Fast Flow (Amersham) and Protein G Sepharose 4 Fast Flow (Amersham). Protein concentrations were determined using Bradford reagent (BioRad). Proteins (20 μg) were subsequently resolved by SDS-PAGE, transferred to nitrocellulose membrane, and immunoblotted with Met (DL-21, Upstate) or β-actin (I-19, Santa Cruz) antibodies. Proteins were visualized by enhanced chemilluminescence (ECL Plus, Amersham).

Results and Discussion

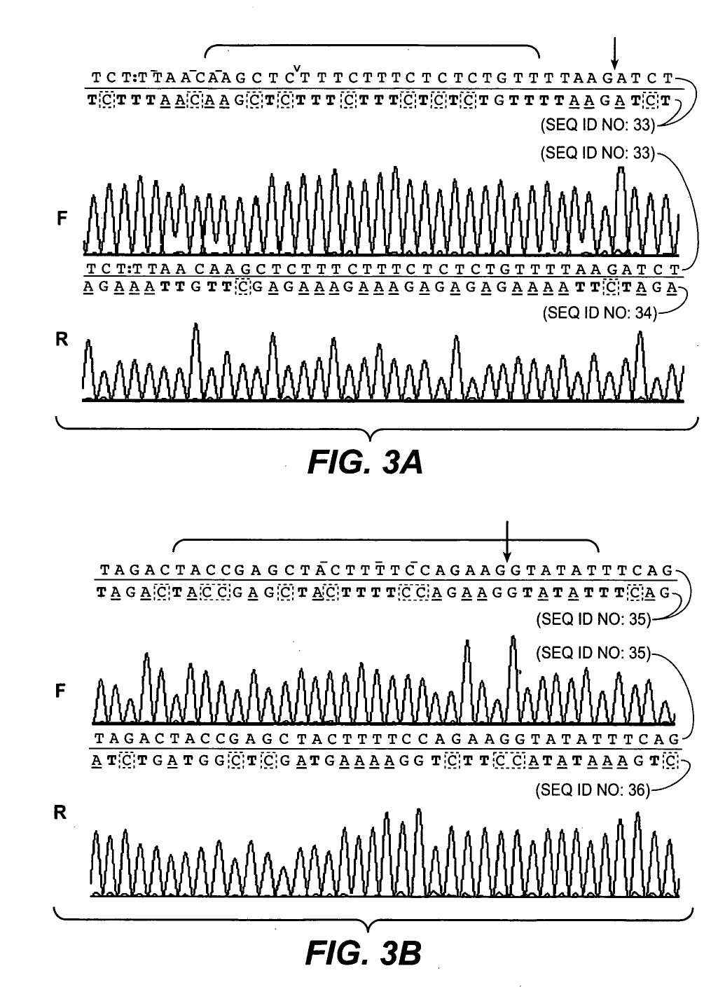

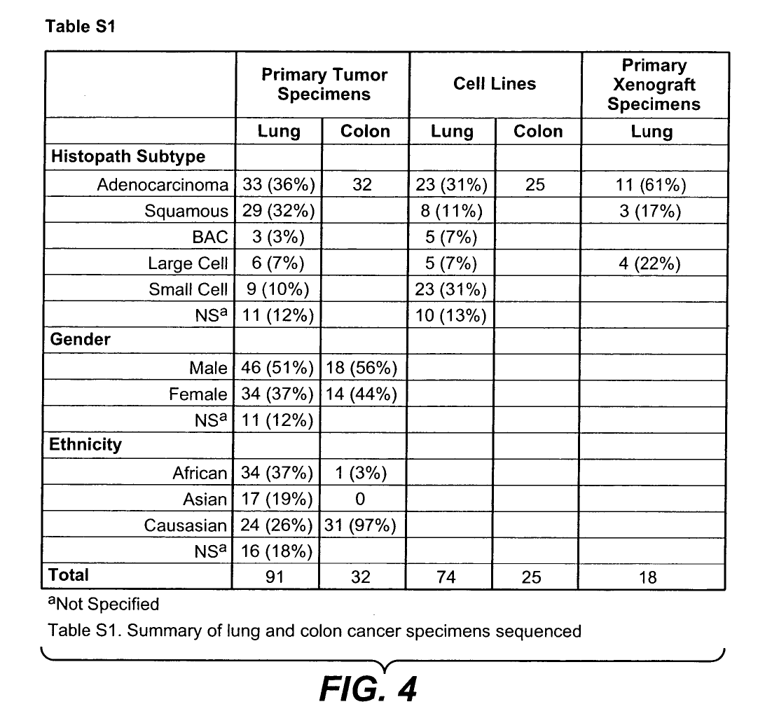

To fully address Met mutations in tumors, we sequenced all coding exons of Met from a panel of lung and colon tumor specimens representing primary tumors, tumor cell lines and primary tumor xenograft models (Table S1 in FIG. 4). In our sequencing effort, we identified somatic heterozygous mutations in primary lung tumor specimens in the intronic regions flanking exon 14 (FIG. 1A, FIG. 2). The mutations mapped exclusively to the intronic region upstream of the 5′ splice site or encompassed the 3′ splice site junction and the surrounding intron at the 3′ end (FIG. 1A). These deletions were tumor specific and were not identified in non-neoplastic lung tissue from the same individuals (FIG. 3). In H596, a non-small cell lung cancer (NSCLC) cell line, we identified a homozygous point mutation in the 3p splice donor site (FIG. 1A). The presence of mutations within the dinucleotidic splice site consensus and the upstream polypyrimidine tract of exon 14, combined with the observation that exon 13 and exon 15 remained in-phase, suggested that a potential Met transcript lacking exon 14 could still produce a functional Met protein. To address this, we first performed RT-PCR amplification of Met RNA from the mutant tumors and cell line. All-three intronic mutations resulted in a transcript of shorter length compared to the wildtype, consistent with deletion of exon 14 (FIG. 1B). We also confirmed the absence of exon 14 by sequencing the RT-PCR products and our results showed an in-frame deletion that removes amino acids L964 through D1010 of Met. Interestingly, the mutant form of the receptor is the most predominantly expressed form, despite the tumor samples being heterozygous for the exon 14 deletion (FIG. 1B), indicating a preferential expression of the variant transcript. This was further confirmed by Western blotting demonstrating the predominant expression of a truncated Met protein (FIG. 1C). Specimens harboring these intronic mutations were wildtype for K-ras, B-raf, EGFR, and HER2 in relevant exons sequenced. Taken together, these results indicate the dominant nature of these Met intronic mutations. Interestingly, a splice variant of Met lacking exon14 has been previously reported in normal mouse tissue, although the functional consequence with respect to tumorigenesis was unclear (20, 21). However, we did not detect expression of this splice variant in any normal human lung specimens examined (data not shown). The lack of this splice variant in normal human tissue has been additionally substantiated, as previously discussed (21). cDNA comprising a splice variant lacking exon 14 has been reported in a primary human NSCLC specimen; however the role of somatic mutagenesis in mediating splicing defects was not assessed, nor was the functional consequence, if any, of any mutant c-met that might have been expressed (22). Since nucleic acids comprising splice variants are not uncommon in cancer cells, the functional relevance of the reported splice variant was unknown.

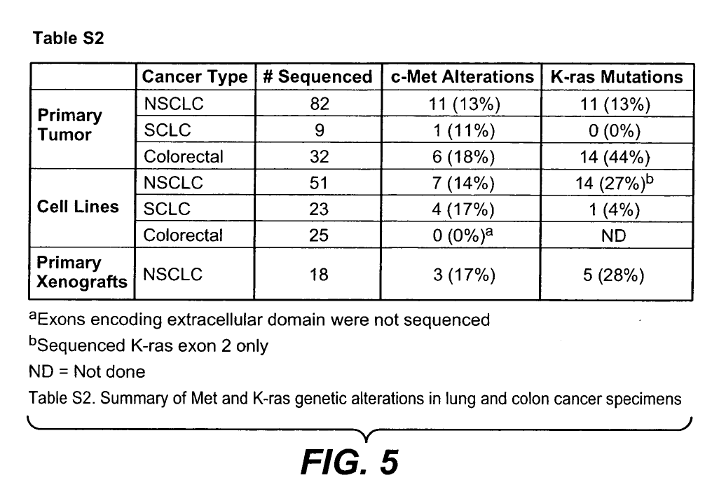

Overall genetic alterations in Met were identified in 13% and 18% of primary lung and colon cancer specimens, respectively, with alterations mapping to the extracellular semaphorin (Sema) domain and the intracellular juxtamembrane and kinase domains (FIG. 1C, Table S2 in FIG. 5, Table S3 in FIG. 6). These alterations were recapitulated in representative cell lines and xenograft models, with additional extracellular domain alterations being identified in lung cancer cell lines. Genetic alterations mapping to the juxtamembrane domain were unique to the lung cancer specimens (6.5%), and were not identified in colon cancer. In addition, juxtamembrane alterations were mutually exclusive with K-ras mutations (Table S2 in FIG. 5). Sequencing DNA from patient-matched, normal adjacent tissue revealed that many of the Met alterations in these primary tumor specimens represented rare polymorphisms (FIG. 1C, Table S3 in FIG. 6). Such polymorphisms included previously reported substitutions in the juxtamembrane domain at amino acid positions R970C and T992I of Met (22, 23). The only additional somatic mutation identified involved an amino acid substitution at position 1108 of the kinase domain that led to a kinase-inactive receptor, as assessed by ectopic expression (data not shown).

The 47 amino acid deletion of exon 14 within the juxtamembrane domain of Met (L964-D1010) removes the Y1003 phosphorylation site necessary for Cb1 binding and down regulation of the activated receptor. To address the loss of this negative regulatory site in mediating Met signaling, we assessed the mechanism of mutant receptor activation. We found that the splice variant protein was functionally able to effect downstream HGF/c-met-associated cellular signaling, and had increased oncogenic potential (data not shown), thus demonstrating for the first time a functional consequence of a c-met splice variant that is associated with tumorigenesis.

In our analysis, activating kinase domain Met mutations were not identified as in RPC and has been noted previously (23). However, kinase domain mutations are found in several receptor tyrosine kinases associated with cancer. Recent characterization of EGFR in lung tumors of patients treated with EGFR inhibitors identified a subset of lung tumors with kinase domain mutations, indicating an important role for EGFR in lung cancer (27-30). Our identification and characterization of a tumor-specific juxtamembrane Met deletion underscores a completely different mechanism of Met activation by delayed receptor down regulation and prolonged downstream signaling. Moreover, these observations strongly suggest that Met plays a significant role in lung cancer. These data imply that similar mutations in receptor down regulation may lead to activation of other oncogenes and merits additional sequence analyses not limited to the kinase domain. Furthermore, the observation of mutations in multiple non-kinase domain positions as described herein suggest that such mutations may also be associated with human lung tumorigenesis, for example by predisposing certain patients to development and/or progression of lung cancers.

Despite the intrinsic nature of aberrant splicing in tumor cells, the involvement of somatic mutations in cis-acting regulatory elements that drive splicing defects is rare. Although germline mutations resulting in splicing defects have been found in several genes, the inactivation of the neurofibromatosis type 1 (NF1) tumor suppressor protein through various mutations provides the only known example of a splicing defect driven by somatic mutagenesis in cancer (31). Our data strongly support the notion that a splicing event driven by somatic mutagenesis is also utilized by lung cancers to activate an oncogenic gene product. The identification of multiple types of intronic mutations that would differentially affect the assembly of the spliceosome, yet selectively exclude exon 14, highlights the relevance of such a mutagenic event in Met, in particular in the context of human lung cancers.

### PARTIAL LIST OF REFERENCES

1. L. Trusolino, P. M. Comoglio, Nat Rev Cancer 2, 289 (April 2002).
2. C. Birchmeier, W. Birchmeier, E. Gherardi, G. F. Vande Woude, Nat Rev Mol Cell Biol 4, 915 (December 2003).
3. C. Ponzetto et al., Cell 77, 261 (Apr. 22, 1994).
4. K. M. Weidner et al., Nature 384, 173 (Nov. 14, 1996).
5. G. Pelicci et al., Oncogene 10, 1631 (Apr. 20, 1995).
6. P. Peschard et al., Mol Cell 8, 995 (November 2001).
7. P. Peschard, N. Ishiyama, T. Lin, S. Lipkowitz, M. Park, J Biol Chem 279, 29565 (Jul. 9, 2004).
8. A. Petrelli et al., Nature 416, 187 (Mar. 14, 2002).
9. K. Shtiegman, Y. Yarden, Semin Cancer Biol 13, 29 (February 2003).
10. M. D. Marmor, Y. Yarden, Oncogene 23, 2057 (Mar. 15, 2004).
11. P. Peschard, M. Park, Cancer Cell 3, 519 (June 2003).
12. J. M. Siegfried et al., Ann Thorac Surg 66, 1915 (December 1998).
13. P. C. Ma et al., Anticancer Res 23, 49 (January-February 2003).
14. B. E. Elliott, W. L. Hung, A. H. Boag, A. B. Tuck, Can J Physiol Pharmacol 80, 91 (February 2002).
15. C. Seidel, M. Borset, H. Hjorth-Hansen, A. Sundan, A. Waage, Med Oncol 15, 145 (September 1998).
16. G. Maulik et al., Cytokine Growth Factor Rev 13, 41 (February 2002).
17. R. Wang, L. D. Ferrell, S. Faouzi, J. J. Maher, J. M. Bishop, J Cell Biol 153, 1023 (May 28, 2001).
18. L. Schmidt et al., Nat Genet 16, 68 (May 1997).
19. M. Jeffers et al., Proc Natl Acad Sci USA 94, 11445 (Oct. 14, 1997).
20. C. C. Lee, K. M. Yamada, J Biol Chem 269, 19457 (Jul. 29, 1994).
21. C. M. Baek, S. H. Jeon, J. J. Jang, B. S. Lee, J. H. Lee, Exp Mol Med 36, 283 (Aug. 31, 2004).
22. P. C. Ma et al., Cancer Res 65, 1479 (Feb. 15, 2005).
23. P. C. Ma et al., Cancer Res 63, 6272 (Oct. 1, 2003).
24. M. Jeffers, G. A. Taylor, K. M. Weidner, S. Omura, G. F. Vande Woude, Mol Cell Biol 17, 799 (February 1997).
25. K. Ohashi et al., Nat Med 6, 327 (March 2000).
26. M. Kong-Beltran, J. Stamos, D. Wickramasinghe, Cancer Cell 6, 75 (July 2004).
27. T. J. Lynch et al., N Engl J Med 350, 2129 (May 20, 2004).
28. R. Sordella, D. W. Bell, D. A. Haber, J. Settleman, Science 305, 1163 (Aug. 20, 2004).
29. W. Pao et al., Proc Natl Acad Sci USA 101, 13306 (Sep. 7, 2004).
30. J. G. Paez et al., Science 304, 1497 (Jun. 4, 2004).
31. E. Serra et al., Hum Genet 108, 416 (May 2001).

1. A prognostic method comprising determining whether a lung cancer sample from a subject comprises a mutation in a nucleic acid sequence encoding human c-met, wherein the mutation results in an amino acid change at position N375, I638, V13, V923, I316 and/or E168.

2. A prognostic method comprising determining whether a lung cancer sample from a subject comprises a mutation in a nucleic acid sequence encoding human c-met, wherein the sequence is mutated in exon 14 and/or its flanking introns, wherein the mutation affects exon splicing.

3. A method of detecting lung cancer in a sample comprising determining whether the sample comprises a mutation in a nucleic acid sequence encoding human c-met, wherein the mutation results in an amino acid change at position N375, I638, V13, V923, I316 and/or E168.

4. A method of detecting lung cancer in a sample comprising determining whether the sample comprises a mutation in a nucleic acid sequence encoding human c-met, wherein the mutation is in exon 14 and/or its flanking introns, wherein the mutation affects exon splicing.

5. A method for distinguishing between non-cancerous and cancerous lung tissue, said method comprising determining whether a sample comprising the lung tissue comprises a mutation in a nucleic acid sequence encoding human c-met, wherein the mutation results in an amino acid change at position N375, I638, V13, V923, I316 and/or E168, wherein detection of the mutation in the sample is indicative of presence of cancerous lung tissue.

6. A method for distinguishing between non-cancerous and cancerous lung tissue, said method comprising determining whether a sample comprising the lung tissue comprises a mutation in a nucleic acid sequence encoding human c-met, wherein the mutation is in exon 14 and/or its flanking introns, wherein the mutation affects exon splicing, wherein detection of the mutation in the sample is indicative of presence of cancerous lung tissue.

7-8. (canceled)

9. A method of identifying a lung cancer that is susceptible to treatment with a c-met inhibitor, said method comprising determining whether a lung cancer sample from a subject comprises a mutation in a nucleic acid sequence encoding human c-met, wherein the mutation results in an amino acid change at position N375, I638, V13, V923, I316 and/or E168.

10. A method of identifying a lung cancer that is susceptible to treatment with a c-met inhibitor, said method comprising determining whether a lung cancer sample from a subject comprises a mutation in a nucleic acid sequence encoding human c-met, whether the sequence is mutated in exon 14 and/or its flanking introns, wherein the mutation affects exon splicing.

11. A method of determining responsiveness of a lung cancer in a subject to treatment with a c-met inhibitor, said method comprising determining whether a lung cancer sample from a subject who has been treated with the c-met inhibitor comprises a mutation in a nucleic acid sequence encoding human c-met, wherein the mutation results in an amino acid change at position N375, I638, V13, V923, I316 and/or E168, wherein absence of the mutated nucleic acid sequence is indicative that the lung cancer is responsive to treatment with the c-met inhibitor.

12. A method of determining responsiveness of a lung cancer in a subject to treatment with a c-met inhibitor, said method comprising determining whether a lung cancer sample from a subject who has been treated with the c-met inhibitor comprises a mutation in a nucleic acid sequence encoding human c-met, whether the sequence is mutated in exon 14 and/or its flanking introns, wherein the mutation affects exon splicing, wherein absence of the mutated nucleic acid sequence is indicative that the lung cancer is responsive to treatment with the c-met inhibitor.

13-19. (canceled)

20. A method of detecting presence of a mutated c-met in lung cancer, the method comprising contacting a sample suspected or known to comprise mutated c-met with an antigen binding agent, wherein binding or lack thereof, of the agent is indicative of presence or absence: of a c-met polypeptide comprising a deletion of at least a portion of exon 14.

21. A method for detecting a cancerous disease state in a lung tissue, said method comprising determining whether a sample from a subject suspected of having lung cancer comprises. a mutation in a nucleic acid sequence encoding human c-met, wherein the mutation results in an amino acid change at position N375, I638, V13, V923, I316 and/or E168, wherein detection of said mutation is indicative of presence of a cancerous disease state in the lung of the subject.

22-28. (canceled)

29. A lung cancer imaging agent, wherein the agent specifically binds c-met comprising a mutation, wherein the agent binds a c-met polypeptide comprising a mutation at position N375, I638, V13, V923, I316 and/or E168 of the protein, or wherein the agent binds a c-met encoding nucleic acid comprising a mutation at a nucleic acid position corresponding to a change in amino acid at position N375, I638, V13, V923, I316 and/or E168.

30. A lung cancer imaging agent, wherein the agent specifically binds c-met polypeptide comprising a deletion of at least a portion of exon 14, or wherein the agent specifically binds c-met encoding nucleic acid that lacks at least a portion of the sequence that encodes exon 14.

31-40. (canceled)


SEQUENCE LISTING (165 sequences)

SEQ ID NO: 1 - protein, 5 aa, Artificial sequence
  Sequence is synthesized
SYWLH

SEQ ID NO: 2 - protein, 17 aa, Artificial sequence
  Sequence is synthesized
MIDPSNSDTRFNPNFKD

SEQ ID NO: 3 - protein, 10 aa, Artificial sequence
  Sequence is synthesized
YGSYVSPLDY

SEQ ID NO: 4 - protein, 17 aa, Artificial sequence
  Sequence is synthesized
KSSQSLLYTSSQKNYLA

SEQ ID NO: 5 - protein, 7 aa, Artificial sequence
  Sequence is synthesized
WASTRES

SEQ ID NO: 6 - protein, 9 aa, Artificial sequence
  Sequence is synthesized
QQYYAYPWT

SEQ ID NO: 7 - protein, 119 aa, Artificial sequence
  Sequence is synthesized
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSS

SEQ ID NO: 8 - protein, 113 aa, Artificial sequence
  Sequence is synthesized
DIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIK

SEQ ID NO: 9 - DNA, 21 bp, Artificial sequence
  Sequence is synthesized
tggttggagagctcatttgga

SEQ ID NO: 10 - DNA, 21 bp, Artificial sequence
  Sequence is synthesized
gcactctcgtcggtgactgtt

SEQ ID NO: 11 - DNA, 18 bp, Artificial sequence
  Sequence is synthesized
tttgccgatttcatgact

SEQ ID NO: 12 - DNA, 28 bp, Artificial sequence
  Sequence is synthesized
cattaaaggagacctcaccatagctaat

SEQ ID NO: 13 - DNA, 22 bp, Artificial sequence
  Sequence is synthesized
cctgatcgagaaaccacaacct

SEQ ID NO: 14 - DNA, 25 bp, Artificial sequence
  Sequence is synthesized
catgaagcgaccctctgatgtccca

SEQ ID NO: 15 - DNA, 45 bp, Homo sapiens
gtagactaccgagctacttttccagaaggtatatttcagtttatt

SEQ ID NO: 16 - DNA, 44 bp, Homo sapiens
gtagactaccgagctacttttccagaagtatatttcagtttatt

SEQ ID NO: 17 - DNA, 11 bp, Homo sapiens
ccagaaggtat

SEQ ID NO: 18 - DNA, 11 bp, Homo sapiens
ccagaagttat

SEQ ID NO: 19 - DNA, 11 bp, Homo sapiens
ggtcttcaata

SEQ ID NO: 20 - DNA, 45 bp, Homo sapiens
tctttaacaagctctttctttctctctgttttaagatctgggcag

SEQ ID NO: 21 - DNA, 23 bp, Homo sapiens
tctttaacttaagatctgggcag

SEQ ID NO: 22 - DNA, 22 bp, Homo sapiens
aagctctttctttctctctgtt

SEQ ID NO: 23 - DNA, 42 bp, Homo sapiens
cgtctttaacaagctctttctttctctctgttttaagatctg

SEQ ID NO: 24 - DNA, 42 bp, Homo sapiens
cgtctttaacttaagatctgggcagtgaattagttcgctacg

SEQ ID NO: 25 - DNA, 42 bp, Homo sapiens; identical to SEQ ID NO: 23

SEQ ID NO: 26 - DNA, 42 bp, Homo sapiens
gtctcctggggcccatgatagccgtctttaacttaagatctg

SEQ ID NO: 27 - DNA, 45 bp, Homo sapiens; identical to SEQ ID NO: 15

SEQ ID NO: 28 - DNA, 17 bp, Homo sapiens
gtagacttcagtttatt

SEQ ID NO: 29 - DNA, 28 bp, Homo sapiens
accgagctacttttccagaaggtatatt

SEQ ID NO: 30 - DNA, 47 bp, Homo sapiens
tctgtagactaccgagctacttttccagaaggtatatttcagtttat

SEQ ID NO: 31 - DNA, 47 bp, Homo sapiens
tctgtagacttcagtttattgttctgagaaatacctatacatatacc

SEQ ID NO: 32 - DNA, 47 bp, Homo sapiens
cccaactacagaaatggtttcaaatgaatctgtagacttcagtttat

SEQ ID NO: 33 - DNA, 39 bp, Homo sapiens
tctttaacaagctctttctttctctctgttttaagatct

SEQ ID NO: 34 - DNA, 39 bp, Homo sapiens
agatcttaaaagagagagaaagaaagagcttgttaaaga

SEQ ID NO: 35 - DNA, 38 bp, Homo sapiens
tagactaccgagctacttttccagaaggtatatttcag

SEQ ID NO: 36 - DNA, 38 bp, Homo sapiens
ctgaaatataccttctggaaaagtagctcggtagtcta

SEQ ID NO: 37 - DNA, 22 bp, Homo sapiens
aagctctttctttctctctgtt

SEQ ID NO: 38 - DNA, 28 bp, Homo sapiens
accgagctacttttccagaaggtatatt

SEQ ID NO: 39 - DNA, 40 bp, Artificial sequence
  Sequence is synthesized
tgtaaaacgacggccagtcagttgggaagctttatttctg

SEQ ID NO: 40 - DNA, 43 bp, Artificial sequence
  Sequence is synthesized
tactactagttgagtaatcgacaccccagtatcgacaaaggac

SEQ ID NO: 41 - DNA, 22 bp, Artificial sequence
  Sequence is synthesized
aaaagtccagttgggaagcttt

SEQ ID NO: 42 - DNA, 23 bp, Artificial sequence
  Sequence is synthesized
ctagttgagtaatcgacaccgtc

SEQ ID NO: 43 - DNA, 39 bp, Artificial sequence
  Sequence is synthesized
tgtaaaacgacggccagtcacccagattgtttcccatgt

SEQ ID NO: 44 - DNA, 43 bp, Artificial sequence
  Sequence is synthesized
ataaatgaagaactgccaggtttccccagtatcgacaaaggac

SEQ ID NO: 45 - DNA, 22 bp, Artificial sequence
  Sequence is synthesized
gctggaacacccagattgtttc

SEQ ID NO: 46 - DNA, 21 bp, Artificial sequence
  Sequence is synthesized
tgaagaactgccaggtttccc

SEQ ID NO: 47 - DNA, 43 bp, Artificial sequence
  Sequence is synthesized
tgtaaaacgacggccagtatcagtgagaaggctaaaggaaacg

SEQ ID NO: 48 - DNA, 43 bp, Artificial sequence
  Sequence is synthesized
gtttttgttacactctacagaggtcccagtatcgacaaaggac

SEQ ID NO: 49 - DNA, 22 bp, Artificial sequence
  Sequence is synthesized
attcgatatcagtgagaaggct

SEQ ID NO: 50 - DNA, 22 bp, Artificial sequence
  Sequence is synthesized
tgttacactctacagaggtcgt

SEQ ID NO: 51 - DNA, 39 bp, Artificial sequence
  Sequence is synthesized
tgtaaaacgacggccagtggtgttcgcacaaagcaagcc

SEQ ID NO: 52 - DNA, 43 bp, Artificial sequence
  Sequence is synthesized
cagagtttttcttataggtcccgaaccagtatcgacaaaggac

SEQ ID NO: 53 - DNA, 21 bp, Artificial sequence
  Sequence is synthesized
tttcggggtgttcgcacaaag

SEQ ID NO: 54 - DNA, 25 bp, Artificial sequence
  Sequence is synthesized
ttcttataggtcccgaagaaaacac

SEQ ID NO: 55 - DNA, 43 bp, Artificial sequence
  Sequence is synthesized
tgtaaaacgacggccagttacacactgaaaggtttcttaccag

SEQ ID NO: 56 - DNA, 43 bp, Artificial sequence
  Sequence is synthesized
agtatcatctttgaccttgtgtctgccagtatcgacaaaggac

SEQ ID NO: 57 - DNA, 25 bp, Artificial sequence
  Sequence is synthesized
gctctgaaaatacacactgaaaggt

SEQ ID NO: 58 - DNA, 25 bp, Artificial sequence
  Sequence is synthesized
catctttgaccttgtgtctgacttt

SEQ ID NO: 59 - DNA, 40 bp, Artificial sequence
  Sequence is synthesized
tgtaaaacgacggccagtccctgctaatctgttattacca

SEQ ID NO: 60 - DNA, 43 bp, Artificial sequence
  Sequence is synthesized
tataaatgttggtcacagtgaaccgccagtatcgacaaaggac

SEQ ID NO: 61 - DNA, 21 bp, Artificial sequence
  Sequence is synthesized
cccacaagccctgctaatctg

SEQ ID NO: 62 - DNA, 21 bp, Artificial sequence
  Sequence is synthesized
atgttggtcacagtgaaccgg

SEQ ID NO: 63 - DNA, 43 bp, Artificial sequence
  Sequence is synthesized
tgtaaaacgacggccagtgcacacatagttgctaagtctagaa

SEQ ID NO: 64 - DNA, 40 bp, Artificial sequence
  Sequence is synthesized
gagtaaatcgacactcagtagtccagtatcgacaaaggac

SEQ ID NO: 65 - DNA, 23 bp, Artificial sequence
  Sequence is synthesized
caggatttgcacacatagttgct

SEQ ID NO: 66 - DNA, 24 bp, Artificial sequence
  Sequence is synthesized
cgacactcagtagtcgatttcgtg

SEQ ID NO: 67 - DNA, 41 bp, Artificial sequence
  Sequence is synthesized
tgtaaaacgacggccagtctgaaatttgtgacccttttccc

SEQ ID NO: 68 - DNA, 40 bp, Artificial sequence
  Sequence is synthesized
ttaacgagttcttcgagtagagccagtatcgacaaaggac

SEQ ID NO: 69 - DNA, 23 bp, Artificial sequence
  Sequence is synthesized
ccagatgctctgaaatttgtgac

SEQ ID NO: 70 - DNA, 25 bp, Artificial sequence
  Sequence is synthesized
gagaacttaatttttcccagaaccg

SEQ ID NO: 71 - DNA, 43 bp, Artificial sequence
  Sequence is synthesized
tgtaaaacgacggccagtgctcaccatttagagttaatgtcac

SEQ ID NO: 72 - DNA, 41 bp, Artificial sequence
  Sequence is synthesized
gaggggaggtcctaggacattatccagtatcgacaaaggac

SEQ ID NO: 73 - DNA, 25 bp, Artificial sequence
  Sequence is synthesized
aaagcagtcagctcaccatttagag

SEQ ID NO: 74 - DNA, 25 bp, Artificial sequence
  Sequence is synthesized
gaggtcctaggacattattgttcat

SEQ ID NO: 75 - DNA, 43 bp, Artificial sequence
  Sequence is synthesized
tgtaaaacgacggccagtcctatgtggtaaggaagattctatc

SEQ ID NO: 76 - DNA, 43 bp, Artificial sequence
  Sequence is synthesized
atgactgttttgaaggaggaaggttccagtatcgacaaaggac

SEQ ID NO: 77 - DNA, 25 bp, Artificial sequence
  Sequence is synthesized
agtacattctcctatgtggtaagga

SEQ ID NO: 78 - DNA, 25 bp, Artificial sequence
  Sequence is synthesized
gaggaaggttttaagtagatgagga

SEQ ID NO: 79 - DNA, 40 bp, Artificial sequence
  Sequence is synthesized
tgtaaaacgacggccagtccacttaggaaccattgagtta

SEQ ID NO: 80 - DNA, 43 bp, Artificial sequence
  Sequence is synthesized
cgacatcttatcagttctccttaacccagtatcgacaaaggac

SEQ ID NO: 81 - DNA, 24 bp, Artificial sequence
  Sequence is synthesized
aggaaattcccacttaggaaccat

SEQ ID NO: 82 - DNA, 22 bp, Artificial sequence
  Sequence is synthesized
cttatcagttctccttaacgtc

SEQ ID NO: 83 - DNA, 39 bp, Artificial sequence
  Sequence is synthesized
tgtaaaacgacggccagtacctgtaatcagtgcaggtga

SEQ ID NO: 84 - DNA, 40 bp, Artificial sequence
  Sequence is synthesized
ttggtacaccgaaagtaccatgccagtatcgacaaaggac

SEQ ID NO: 85 - DNA, 22 bp, Artificial sequence
  Sequence is synthesized
gcctctgacctgtaatcagtgc

SEQ ID NO: 86 - DNA, 25 bp, Artificial sequence
  Sequence is synthesized
acaccgaaagtaccatggactctgt

SEQ ID NO: 87 - protein, 43 aa, Artificial sequence
  Sequence is synthesized
TGTAAAACGACGGCCAGTCGTGTTTCCAGAAATGTGTAGTCTA

SEQ ID NO: 88 - DNA, 43 bp, Artificial sequence
  Sequence is synthesized
cacttatgttaacaacatgaaccggccagtatcgacaaaggac

SEQ ID NO: 89 - DNA, 22 bp, Artificial sequence
  Sequence is synthesized
tgcatgtatcgtgtttccagaa

SEQ ID NO: 90 - DNA, 22 bp, Artificial sequence
  Sequence is synthesized
gttaacaacatgaaccggtaac

SEQ ID NO: 91 - DNA, 40 bp, Artificial sequence
  Sequence is synthesized
tgtaaaacgacggccagtggcctgtgtttgcagtatattt

SEQ ID NO: 92 - DNA, 43 bp, Artificial sequence
  Sequence is synthesized
ttcgagtaaatacacacccaaaaccccagtatcgacaaaggac

SEQ ID NO: 93 - DNA, 21 bp, Artificial sequence
  Sequence is synthesized
caacatggcctgtgtttgcag

SEQ ID NO: 94 - DNA, 21 bp, Artificial sequence
  Sequence is synthesized
cacacccaaaaccgagtagtt

SEQ ID NO: 95 - DNA, 41 bp, Artificial sequence
  Sequence is synthesized
tgtaaaacgacggccagtcaaagtgctacaacctgtgtagt

SEQ ID NO: 96 - DNA, 43 bp, Artificial sequence
  Sequence is synthesized
attctaacagcagctaagaacacacccagtatcgacaaaggac

SEQ ID NO: 97 - DNA, 21 bp, Artificial sequence
  Sequence is synthesized
gggacccaaagtgctacaacc

SEQ ID NO: 98 - DNA, 22 bp, Artificial sequence
  Sequence is synthesized
acatcaggtattttgggtactc

SEQ ID NO: 99 - DNA, 44 bp, Artificial sequence
  Sequence is synthesized
tgtaaaacgacggccagtgtcattacagtttaagattgtcgtcg

SEQ ID NO: 100 - DNA, 44 bp, Artificial sequence
  Sequence is synthesized
ggagacggttcattcataaactgtgtccagtatcgacaaaggac

SEQ ID NO: 101 - DNA, 36 bp, Artificial sequence
  Sequence is synthesized
gaagttaccttaagaacacagtcattacagtttaag

SEQ ID NO: 102 - DNA, 32 bp, Artificial sequence
  Sequence is synthesized
cattcataaactgtgttttaatgtaccgagaa

SEQ ID NO: 103 - DNA, 41 bp, Artificial sequence
  Sequence is synthesized
tgtaaaacgacggccagtaaagctcttcctgtttcagtccc

SEQ ID NO: 104 - DNA, 43 bp, Artificial sequence
  Sequence is synthesized
aatagaaaccggaaacgtctaatccccagtatcgacaaaggac

SEQ ID NO: 105 - DNA, 25 bp, Artificial sequence
  Sequence is synthesized
gtctaaggaaatgagggtaaaaagc

SEQ ID NO: 106 - DNA, 22 bp, Artificial sequence
  Sequence is synthesized
aaaccggaaacgtctaatcctt

SEQ ID NO: 107 - DNA, 43 bp, Artificial sequence
  Sequence is synthesized
tgtaaaacgacggccagtcctacgtacctatagtggtattgtt

SEQ ID NO: 108 - DNA, 43 bp, Artificial sequence
  Sequence is synthesized
ttgcaactaaatgtgaaaggggaacccagtatcgacaaaggac

SEQ ID NO: 109 - DNA, 21 bp, Artificial sequence
  Sequence is synthesized
ggtgaaatgtgttgcatctac

SEQ ID NO: 110 - DNA, 23 bp, Artificial sequence
  Sequence is synthesized
actaaatgtgaaaggggaacacc

SEQ ID NO: 111 - DNA, 43 bp, Artificial sequence
  Sequence is synthesized
tgtaaaacgacggccagtggtttaccatttcattgctcttcct

SEQ ID NO: 112 - DNA, 40 bp, Artificial sequence
  Sequence is synthesized
tagggaagttttatccggacgaccagtatcgacaaaggac

SEQ ID NO: 113 - DNA, 25 bp, Artificial sequence
  Sequence is synthesized
gctaactgtgtggtttaccatttca

SEQ ID NO: 114 - DNA, 24 bp, Artificial sequence
  Sequence is synthesized
aagttttatccggacgagactcag

SEQ ID NO: 115 - DNA, 39 bp, Artificial sequence
  Sequence is synthesized
tgtaaaacgacggccagttcaatggggcacactttttgt

SEQ ID NO: 116 - DNA, 43 bp, Artificial sequence
  Sequence is synthesized
agaacgttcgtttttcaaacaggtgccagtatcgacaaaggac

SEQ ID NO: 117 - DNA, 21 bp, Artificial sequence
  Sequence is synthesized
gatctgtcaatggggcacact

SEQ ID NO: 118 - DNA, 25 bp, Artificial sequence
  Sequence is synthesized
tcgtttttcaaacaggtgtctctga

SEQ ID NO: 119 - DNA, 43 bp, Artificial sequence
  Sequence is synthesized
tgtaaaacgacggccagtctgaagccacttgtttaatctgtag

SEQ ID NO: 120 - DNA, 43 bp, Artificial sequence
  Sequence is synthesized
tttacctgaacagactatgcatgtgccagtatcgacaaaggac

SEQ ID NO: 121 - DNA, 25 bp, Artificial sequence
  Sequence is synthesized
ggatttcaaatactgaagccacttg

SEQ ID NO: 122 - DNA, 25 bp, Artificial sequence
  Sequence is synthesized
ctgaacagactatgcatgtgtaaca

SEQ ID NO: 123 - DNA, 43 bp, Artificial sequence
  Sequence is synthesized
tgtaaaacgacggccagtccaagacctactgatttcctttcat

SEQ ID NO: 124 - DNA, 41 bp, Artificial sequence
  Sequence is synthesized
acggaagtttcccagagaatgtcccagtatcgacaaaggac

SEQ ID NO: 125 - DNA, 25 bp, Artificial sequence
  Sequence is synthesized
caagtttagttaccaagacctactg

SEQ ID NO: 126 - DNA, 25 bp, Artificial sequence
  Sequence is synthesized
agtttcccagagaatgtcgtacaga

SEQ ID NO: 127 - protein, 43 aa, Artificial sequence
  Sequence is synthesized
TGTAAAACGACGGCCAGTAGTCTTTCTGTACCTCTTACGTTCT

SEQ ID NO: 128 - DNA, 43 bp, Artificial sequence
  Sequence is synthesized
ctggaaattttccggtagctataagccagtatcgacaaaggac

SEQ ID NO: 129 - DNA, 25 bp, Artificial sequence
  Sequence is synthesized
cccttgtaagtagtctttctgtacc

SEQ ID NO: 130 - DNA, 25 bp, Artificial sequence
  Sequence is synthesized
ttttccggtagctataagaaacgag

SEQ ID NO: 131 - DNA, 43 bp, Artificial sequence
  Sequence is synthesized
tgtaaaacgacggccagtgtactggtggagtatttgatagtgt

SEQ ID NO: 132 - DNA, 40 bp, Artificial sequence
  Sequence is synthesized
gtctatttccaaagagactggtccagtatcgacaaaggac

SEQ ID NO: 133 - DNA, 22 bp, Artificial sequence
  Sequence is synthesized
gcagtcaactggaattttcatg

SEQ ID NO: 134 - DNA, 25 bp, Artificial sequence
  Sequence is synthesized
ccaaagagactggtaaaagtactca

SEQ ID NO: 135 - protein, 46 aa, Artificial sequence
  Sequence is synthesized
TGTAAAACGACGGCCAGTGTAAAAGGTGCACTGTAATAATCCAGAC

SEQ ID NO: 136 - DNA, 44 bp, Artificial sequence
  Sequence is synthesized
ctcagaaacgattacggtacgtatatccagtatcgacaaaggac

SEQ ID NO: 137 - DNA, 21 bp, Artificial sequence
  Sequence is synthesized
tttgaagtaaaaggtgcactg

SEQ ID NO: 138 - DNA, 27 bp, Artificial sequence
  Sequence is synthesized
cgattacggtacgtatattataaatta

SEQ ID NO: 139 - DNA, 39 bp, Artificial sequence
  Sequence is synthesized
tgtaaaacgacggccagtccttaggtgcggctccacagc

SEQ ID NO: 140 - DNA, 40 bp, Artificial sequence
  Sequence is synthesized
cgagtagaggtgtaggatttacccagtatcgacaaaggac

SEQ ID NO: 141 - DNA, 25 bp, Artificial sequence
  Sequence is synthesized
gcaatatcagccttaggtgcggctc

SEQ ID NO: 142 - DNA, 26 bp, Artificial sequence
  Sequence is synthesized
gtgtaggatttacaagtgaaagatac

SEQ ID NO: 143 - DNA, 40 bp, Artificial sequence
  Sequence is synthesized
tgtaaaacgacggccagtcagccataagtcctcgacgtgg

SEQ ID NO: 144 - DNA, 42 bp, Artificial sequence
  Sequence is synthesized
caaattgtgtacgtcccctcctacccagtatcgacaaaggac

SEQ ID NO: 145 - DNA, 24 bp, Artificial sequence
  Sequence is synthesized
ctaacgttcgccagccataagtcc

SEQ ID NO: 146 - DNA, 26 bp, Artificial sequence
  Sequence is synthesized
ggtctgtaagacccactcgagcgtcg

SEQ ID NO: 147 - DNA, 43 bp, Artificial sequence
  Sequence is synthesized
tgtaaaacgacggccagtgcataaggtaatgtacttagggtga

SEQ ID NO: 148 - DNA, 40 bp, Artificial sequence
  Sequence is synthesized
cctttataagtgacaagcgtagccagtatcgacaaaggac

SEQ ID NO: 149 - DNA, 24 bp, Artificial sequence
  Sequence is synthesized
tccctctcaggcataaggtaatgt

SEQ ID NO: 150 - DNA, 25 bp, Artificial sequence
  Sequence is synthesized
aaaactaatcaaagtcctgaggagg

SEQ ID NO: 151 - DNA, 40 bp, Artificial sequence
  Sequence is synthesized
tgtaaaacgacggccagtctaacacatttcaagccccaaa

SEQ ID NO: 152 - DNA, 43 bp, Artificial sequence
  Sequence is synthesized
gacgactcaatgatctttcagtaacccagtatcgacaaaggac

SEQ ID NO: 153 - DNA, 25 bp, Artificial sequence
  Sequence is synthesized
tcacctcatcctaacacatttcaag

SEQ ID NO: 154 - DNA, 25 bp, Artificial sequence
  Sequence is synthesized
gatctttcagtaacttccagagttg

SEQ ID NO: 155 - DNA, 39 bp, Artificial sequence
  Sequence is synthesized
tgtaaaacgacggccagtgccatggctgtggtttgtgat

SEQ ID NO: 156 - DNA, 39 bp, Artificial sequence
  Sequence is synthesized
ccgatcctacccctgagaacgccagtatcgacaaaggac

SEQ ID NO: 157 - DNA, 21 bp, Artificial sequence
  Sequence is synthesized
cttcacaggctgtgggccatg

SEQ ID NO: 158 - DNA, 22 bp, Artificial sequence
  Sequence is synthesized
ggtccgggagggtcttccagat

SEQ ID NO: 159 - DNA, 25 bp, Artificial sequence
  Sequence is synthesized
gaccctgaagcagttaaaggtgaag

SEQ ID NO: 160 - DNA, 24 bp, Artificial sequence
  Sequence is synthesized
gttgtcaggtgtgaaacaggttac

SEQ ID NO: 161 - DNA, 18 bp, Artificial sequence
  Sequence is synthesized
ctgtgaagcgcgccgtga

SEQ ID NO: 162 - DNA, 25 bp, Artificial sequence
  Sequence is synthesized
gtgtcctaactaacgaccacaacag

SEQ ID NO: 163 - DNA, 28 bp, Homo sapiens
ttttccagaaggtatatttcagtttatt

SEQ ID NO: 164 - DNA, 45 bp, Homo sapiens; identical to SEQ ID NO: 20

SEQ ID NO: 165 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKAMKYLASKKFVHRDLAARNCMLDEKFTVKVADFGLARDMYDKEYYSVHNKTGAKLPVKWMALESLQTQKFTTKSDVWSFGVVLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS